(19) United States
(12) Patent Application Publication
(10) Pub. No.: US 2024/0115514 A1
(43) Pub. Date: Apr. 11, 2024

(54) Macrophage Targeted Immunotherapeutics

(71) Applicant: The General Hospital Corporation, Boston, MA (US)

(72) Inventors: Christopher Rodell, Jamaica Plain, MA (US); Ralph Weissleder, Peabody, MA (US)

(21) Appl. No.: 18/215,309

(22) Filed: Jun. 28, 2023

Related U.S. Application Data
(63) Continuation of application No. 16/757,119, filed on Apr. 17, 2020, now abandoned.
(60) Provisional application No. 62/574,942, filed on Oct. 20, 2017.

(51) Int. Cl. A61K 9/51; A61K 31/4745; A61K 47/54; A61K 47/65; A61P 35/00; C07K 16/28

(52) U.S. Cl. CPC A61K 9/5161; A61K 31/4745; A61K 47/54; A61K 47/65; A61P 35/00; C07K 16/2818; A61K 45/06

(57) ABSTRACT

Disclosed herein are nanoparticles that include one or more cyclodextrin moieties crosslinked by a linker. The cyclodextrin moieties can complex therapeutic (e.g., anticancer) agents, and can be used to treat diseases such as cancer.

### CROSS-REFERENCE TO RELATED APPLICATIONS

This application is a continuation of U.S. patent application Ser. No. 16/757,119, filed Apr. 17, 2020, which is a § 371 National Stage Application of PCT/US2018/056780, filed Oct. 19, 2018, which claims the benefit of U.S. Provisional Application Ser. No. 62/574,942, filed Oct. 20, 2017, the disclosure of which is incorporated herein by reference in its entirety.

### FEDERALLY SPONSORED RESEARCH OR DEVELOPMENT

This invention was made with Government support under Grant Nos. T32CA079443; R01CA206890; U01CA206997; and R01HL131495, awarded by the National Institute of Health. The Government has certain rights in the invention.

### TECHNICAL FIELD

Provided herein are nanoparticles useful for binding therapeutic agents (e.g., anticancer agents). Also provided are methods of using the nanoparticles to treat cancer.

### BACKGROUND

Chemotherapy, targeted therapy, radiation therapy, and hormonal therapy are commonly used methods in the prevention, diagnosis, and treatment of cancer (see, e.g., A. Gadducci, S. Cosio, A. R. Genazzani, Crit. Rev. Oncol. Hematol. 2006, 58, 242-256). Immune cells play roles in regulating tumor growth, and as such, can be harnessed for anticancer therapy. For example, immunotherapies targeting T-cell immune functions have improved survival rates of cancer patients. The tumor microenvironment (TME) includes a diverse set of host cell types that can be therapeutically targeted, including tumor-associated macrophages (TAMs), TAMs are abundant immune cells in the tumor stroma in a broad range of cancers, and a high abundance of these cells in tumors can be associated with a poor clinical outcome.

### SUMMARY

Provided herein is a nanoparticle, comprising at least two host macrocycles, wherein the at least two host macrocycles are covalently crosslinked by a linker, wherein the linker comprises a moiety of Formula (I):

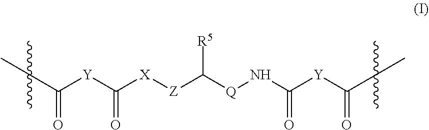

wherein:

Q is selected from a bond or methylene;

X is selected from O, S, and NR¹;

each Y is independently selected from C₁₋₁₀ alkylene optionally substituted with one or more R²;

Z is selected from A-B, wherein A is selected from a bond and C₁₋₁₀ alkylene, and B is selected from C₁₋₁₀ arylene, 3-10 membered heteroarylene, 3-10 membered heterocycloalkyl, and C₃₋₁₀ cycloalkyl;

wherein A is optionally substituted with one or more R³, and B is optionally substituted with one or more R⁴;

R¹ is selected from H and C₁₋₃ alkyl;

each R² is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

each R³ is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

each R⁴ is independently selected from C₁₋₁₀ arylene, 3-10 membered heteroarylene, 3-10 membered heterocycloalkyl, C₃₋₁₀ cycloalkyl, hydroxy, halo, CN, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and

R⁵ is selected from H, C₁-C₆ alkyl, CO₂H, C₁₋₁₀ arylene, 3-10 membered heteroarylene, 3-10 membered heterocycloalkyl, C₃₋₁₀ cycloalkyl, hydroxy, halo, CN, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl.

In some embodiments, R⁵ is CO₂H. In some embodiments, Q is a bond. In some embodiments, each Y is ethylene. In some embodiments, X is NH. In some embodiments, Z is n-butylene.

In some embodiments, the at least two host macrocycles comprise less than 1×10⁹ host macrocycles. In some embodiments, the at least two host macrocycles comprise less than 5×10⁶ host macrocycles. In some embodiments, the at least two host macrocycles comprise less than 5000 host macrocycles.

In some embodiments, at least one of the at least two host macrocycles is selected from the group consisting of: cyclodextrin, pillar[n]arenes, calix[n]arenes, and cucurbit[n]urils. In some embodiments, at least two of the at least two host macrocycles are selected from the group consisting of: cyclodextrin, pilllar[n]arenes, calix[n]arenes, and cucurbit[n]urils. In some embodiments, the at least two host macrocycles comprise at least two cyclodextrins.

In some embodiments, each cyclodextrin comprises α-cyclodextrin, β-cyclodextrin, γ-cyclodextrin, 2-hydroxypropyl-α-cyclodextrin, 2-hydroxypropyl-β- cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, or an aminated cyclodextrin, In some embodiments, each cyclodextrin comprises β-cyclodextrin.

In some embodiments, the nanoparticle comprises at least one linear or branched polymer. In some embodiments, the at least one polymer is selected from the group consisting of: a dextran derivative, a hyaluronic acid derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative, a oligo(poly(ethylene glycol)fumarate) derivative, a poly(vinyl alcohol) derivative, and a poly(vinyl acid) derivative.

In some embodiments, the nanoparticle comprises at least one therapeutic agent. In some embodiments, the therapeutic agent forms a host-guest complex with at least one of the host macrocycles. In some embodiments, the at least one therapeutic agent comprises an anticancer or immunomodulating agent. In some embodiments, the at least one therapeutic agent comprises an anticancer agent. In some embodiments, the anticancer agent is a toll-like receptor (TLR) agonist. In some embodiments, the anticancer agent is a TLR3, TLR4, TLR 7/8, or TLR9 agonist.

In some embodiments, one or more of the at least one therapeutic agents is selected from the group consisting of: GW2580, CEP32496, BLZ945, OS930, PLX3397, dasatinib, sunitinib, ABT869, imatinib, foretinib, XL228, gefitinib, PD0325901, trametinib, bentamapimod, dabrafenib, vemurafinib, crizotinib, UNC2025, indoximod, celecoxib, rapamycin, NIK12192, trichostatin A, MET151, TMP195, BYL719, GDC0941, BKM120, imiquimod, gardiquimod, resiquimod (R848), motolimod, and GS9620. In some embodiments, one or more of the at least one therapeutic agents is a compound selected from the group consisting of: imiquimod, indoximod, gardiquimod, motolimod, and resiquimod (R848). In some embodiments, the anticancer agent is resiquimod (R848).

In some embodiments, the nanoparticle comprises two or more therapeutic agents, wherein one of the two or more therapeutic agents improves the efficacy of one or more of the other therapeutic agents.

In some embodiments, the nanoparticle further comprises an imaging agent. In sonic embodiments, the imaging agent comprises a magnetic resonance imaging (MRI) agent, a positron emission tomography (PET) agent, a single-photon emission computed tomography (SPECT) agent, or a near-infrared fluorophore. In some embodiments, the near-infrared fluorophore is selected from the group consisting of Vivo Tag 680-XL, ZW800-1C, ZW800-1, ZW800-3C, ZW700-1, indocyanine green (ICG), Cy5, Cy 5.5, Cy₇, Cy7.5, IRDye800-CW (CW800), BODIPY 630, and ZWCC.

In some embodiments, the at least one therapeutic agent is conjugated with a fluorescent dye.

In some embodiments, the at least one therapeutic agent is conjugated with adamantane.

In some embodiments, the stoichiometric ratio of the cyclodextrin to the therapeutic agent is from about 100:1 to about 1:100. In some embodiments, the stoichiometric ratio of the cyclodextrin to the therapeutic agent is about 1:1. In some embodiments, the stoichiometric ratio of the cyclodextrin to the therapeutic agent is about 1.1:1.

In some embodiments, the equilibrium binding constant (K_{D}) of the at least one therapeutic agent to the host macrocycle is from about 1×10⁻¹² M to about 0.1 M. In some embodiments, the equilibrium binding constant (K_{D}) of the at least one therapeutic agent to the cyclodextrin is from about 5.5 mM to about 7.2 mM. In some embodiments, the equilibrium binding constant (K_{D}) of the at least one therapeutic agent to the cyclodextrin is about 6.3 mM.

In some embodiments, the half-life of the therapeutic agent in vivo after release from the nanoparticle is from about 45 minutes to about 90 minutes. In some embodiments, the half-life of the therapeutic agent in vivo after release from the nanoparticle is about 62 minutes.

In some embodiments, the nanoparticle has an overall negative charge. In some embodiments, the nanoparticle has a zeta potential of from about −5 mV to about −50 mV. In some embodiments, the nanoparticle has a zeta potential of about −10 mV

In some embodiments, the average molecular weight of the nanoparticle is from about 1,500 g/mol to about 5×10¹¹ g/mol. In some embodiments, the average molecular weight of the nanoparticle is from about 15×10³ g/mol to about 20×10⁶ g/mol. In some embodiments, the average molecular weight of the nanoparticle is about 20×10⁶ g/mol.

In some embodiments, the nanoparticie comprises an average of from about 10 to about 10,000 cyclodextrins. In some embodiments, the nanoparticle comprises an average of from about 100 to about 2,000 cyclodextrins. In some embodiments, the nanoparticle comprises an average of about 1,000 cyclodextrins.

In some embodiments, the average hydrodynamic diameter of the nanoparticle is from about 10 nm to about 1000 nm. In some embodiments, the average hydrodynamic diameter of the nanoparticle is from about 10 nm to about 70 nm. In some embodiments, the average hydrodynamic diameter of the nanoparticle is from about 20 nm to about 60 nm. In some embodiments, the average hydrodynamic diameter of the nanoparticle is about 50 nm, In some embodiments, the average hydrodynamic diameter of the nanoparticle is about 30 nm.

Also provided herein is a nanoparticle, comprising:

at least two cyclodextrins, wherein the at least two cyclodextrins are covalently crosslinked by a linker, and wherein the linker comprises a moiety of Formula (I):

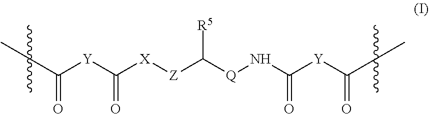

wherein:

Q is selected from a bond or methylene;

X is selected from O, S, and NR¹;

each Y is independently selected from C₁₋₁₀ alkylene optionally substituted with one or more R²;

Z is selected from A-B, wherein A is selected from a bond and C₁₋₁₀ alkylene, and B is selected from C₁₋₁₀ arylene, 3-10 membered heteroarylene, 3-10 membered heterocycloalkyl, and C₃₋₁₀ cycloalkyl;

wherein A is optionally substituted with one or more R³, and B is optionally substituted with one or more R⁴;

R¹ is selected from H and C₁₋₃ alkyl;

each R¹ is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

each R³ is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocydoalkyl), and NHCOC₂-C₆ alkynyl;

each R⁴ is independently selected from C₁₋₁₀ arylene, 3-10 membered heteroarylene, 3-10 membered heterocycloalkyl, C₃₋₁₀ cycloalkyl, hydroxy, halo, CN, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and

R³ is selected from H, C₁-C₆ alkyl, CO₂H, C₁₋₁₀ arylene, 3-10 membered heteroarylene, 3-10 membered heterocycloalkyl, C₃₋₁₀ cycloalkyl, hydroxy, halo, CN, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and a therapeutic agent.

In some embodiments, R⁵ is CO₂H. In some embodiments, Q is a bond. In some embodiments, each Y is ethylene. In some embodiments, X is NH. In some embodiments, Z is n-butylene.

In some embodiments, the at least two host macrocycles comprise less than 1×10⁹ host macrocycles. In some embodiments, the at least two host macrocycles comprise less than 5×10⁶ host macrocycles. In some embodiments, the at least two host macrocycles comprise less than 5000 host macrocycles.

In some embodiments, each cyclodextrin comprises α-cyclodextrin, β-cyclodextrin, γ-cyclodextrin, 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, or an aminated cyclodextrin. In some embodiments, each cyclodextrin comprises β-cyclodextrin.

In some embodiments, the linker comprises L-lysine.

In some embodiments, the nanoparticle comprises at least one linear or branched polymer. In some embodiments, the at least one polymer is selected from the group consisting of: a dextran derivative, a hyaluronic acid derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative; a oligo(poly(ethylene glyco)fumarate) derivative, a poly(vinyl alcohol) derivative, and a poly(vinyl acid) derivative.

In some embodiments, the therapeutic agent forms a host-guest complex with at least one of the cyclodextrins.

In some embodiments, the at least one therapeutic agent comprises an anticancer agent. In some embodiments, the anticancer agent is a toll-like receptor (TLR) agonist. In some embodiments, the anticancer agent is a TLR 7/8 agonist.

In some embodiments, one or more of the at least one therapeutic agents is selected from the group consisting of: GW2580, CEP32496, BLZ945, OSI930, PLX3397, dasatinib, sunitinib, ABT869, imatinib, foretinib, XL228, gefitinib, PD0325901, trametinib, bentamapimod, dabrafenib, vemurafinib, critzotinib, UNC2025, indoximod, celecoxib, rapamycin, NIK12192, trichostatin A, IBET151, TMP195, BYL719, GDC0941, BKM120, resiquimod (R848), motolimod, GS9620, and a compound comprising an imidazoquinoline. In some embodiments, one or more of the at least one therapeutic agents is a compound selected from the group consisting of: imiquimod, indoximod, gardiquimod, motolimod, or resiquimod (R848), In sonic embodiments, one or more of the at least one therapeutic agents is resiquimod (R848).

In sonic embodiments, the nanoparticle comprises two or more therapeutic agents, wherein one of the two or more therapeutic agents improves the efficacy of one or more of the other therapeutic agents.

In some embodiments, the nanoparticle further comprises an imaging agent.

In some embodiments, the imaging agent comprises a magnetic resonance imaging (MRI) agent, a positron emission tomography (PET) agent, a single-photon emission computed tomography (SPECT) agent, or a near-infrared fluorophore. In some embodiments, the near-infrared fluorophore is selected from the group consisting of Vivi Tag 680-XL, ZW800-1C, ZW800-1, ZW800-3C, ZW700-1, indocyanine green (ICG), Cy5, Cy5.5, Cy7, Cy7.5, IRDye800-CW (CW800), BODIPY 630, and ZWCC.

In some embodiments, the at least one therapeutic agent is conjugated with a fluorescent dye.

In some embodiments, the at least one therapeutic agent is conjugated with adamantane.

In some embodiments, the stoichiometric ratio of the cyclodextrin to the therapeutic agent is from about 100:1 to about 1:100. In some embodiments, the stoichiometric ratio of the cyclodextrin to the therapeutic agent is about 1:1. In some embodiments, the stoichiometric ratio of the cyclodextrin to the therapeutic agent is about 1.1:1.

In some embodiments, the equilibrium binding constant (K_{D}) of the at least one therapeutic agent to the cyclodextrin is from about 1×10⁻¹² M to about 0.1 M. In some embodiments, the equilibrium binding constant (K_{D}) of the at least one therapeutic agent to the cyclodextrin is from about 5.5 mM to about 7.2 mM. In some embodiments, the equilibrium binding constant (K_{D}) of the at least one therapeutic agent to the cyclodextrin is about 6.3 mM.

In some embodiments, the half-life of the therapeutic agent in vivo after release from the nanoparticle is from about 45 minutes to about 90 minutes. In some embodiments, the half-life of the therapeutic agent in vivo after release from the nanoparticle is about 62 minutes.

In sonic embodiments, the nanoparticle has an overall negative charge. In some embodiments, the nanoparticle has a zeta potential of from about −5 mV to about −15 mV. In some embodiments, the nanoparticle has a zeta potential of about −10 mV.

In some embodiments, the average molecular weight of the nanoparticle is from about 1,500 g/mol to about 5×10¹¹ g/mol. In some embodiments, the average molecular weight of the nanoparticle is from about 15×10³ g/mol to about 20×10⁶ g/mol. In some embodiments, the average molecular weight of the nanoparticle is about 20×10⁶ g/mol.

In some embodiments, the nanoparticle comprises an average of from about 10 to about 10,000 cyclodextrins. In some embodiments, the nanoparticle comprises an average of from about 100 to about 2,000 cyclodextrins, In some embodiments, the nanoparticle comprises an average of about 1,000 cyclodextrins.

In some embodiments, the average hydrodynamic diameter of the nanoparticle is from about 10 nm to about 1000 nm. In some embodiments, the average hydrodynamic diameter of the nanoparticle is from about 10 nm to about 70 nm. In some embodiments, the average hydrodynamic diameter of the nanoparticle is from about 20 nm to about 60 nm. In some embodiments, the average hydrodynamic diameter of the nanoparticle is about 50 nm. In some embodiments, the average hydrodynamic diameter of the nanoparticle is about 30 nm.

Also provided herein is a pharmaceutical composition comprising any of the foregoing nanoparticles that comprise a therapeutic agent, and a pharmaceutically, acceptable excipient.

Also provided herein is a method of treating cancer in a patient, the method comprising administering a therapeutically effective amount of any of the foregoing nanoparticles that comprise a therapeutic agent, or the foregoing pharmaceutical composition, to the patient.

In some embodiments, the cancer comprises a tumor-associated macrophage, and wherein the phenotype of the macrophage is M2. In some embodiments, the treating further comprises converting the phenotype of the macrophage from M2 to M1.

In some embodiments, the cancer is selected from the group consisting of Ewing sarcoma, osteosarcoma, glioblastoma, meningioma, oligodendrial cancer, melanoma metastasis, melanoma primary, breast cancer, gastric cancer, germ cell tumors, astrocytoma, ovarian cancer, lung large cell carcinoma, lung adenocarcinoma, lung squamous cell carcinoma, colon cancer, head and neck cancer, bladder cancer, thyroid cancer, liver cancer, pancreas cancer, kidney cancer, cervical cancer, testicular cancer, prostate cancer, and bone cancer.

In some embodiments, the cancer is metastatic.

In some embodiments, the uptake of the nanoparticle is higher into tumor associated macrophages than into any other organ or tissue type in the subject after administration.

In sonic embodiments, less than 20 mol % of the therapeutic agent is released prior to uptake of the nanoparticle into tumor macrophage cells. In some embodiments, less than 10 mol % of the nanoparticle is released prior to uptake of the nanoparticle into tumor macrophage cells. In some embodiments, less than 5 mol % of the nanoparticle is released prior to uptake of the nanoparticle into tumor macrophage cells. In some embodiments, less than 1 mol % of the nanoparticle is released prior o uptake of the nanoparticle into cancer cells.

In some embodiments, the nanoparticle or composition is administered intravenously, intraarterially, intratumorally, subcutaneously, or intraperitoneally.

In some embodiments, the method further comprises administering an additional therapeutic agent that improves the efficacy of the nanoparticle. In some embodiments, the additional therapeutic agent is a PD-1 antibody, a CTLA-4 antibody, a PD-L1 antibody, an IDO inhibitor, a CSF-1R inhibitor, kinase inhibitor, an HDAC inhibitor, a PI3K inhibitor, a MerTK inhibitor, or an Ax1 inhibitor. In some embodiments, the additional therapeutic agent is a PD-1 antibody. In some embodiments, the PD-1 antibody is selected from the group consisting of: nivolumab, pembrolizumab, pidilizumab, BMS-936559, atezolizumab, and avelumab.

In some embodiments, further comprising treating the patient with radiation, chemotherapy, antibody checkpoint therapy, immunotherapy, or any combination thereof.

In some embodiments, the treating comprises slowing the formation of cancer cells. In some embodiments, the treating comprises preventing the formation of cancer cells. In some embodiments, the treating comprises killing cancer cells.

In some embodiments, the patient is a human.

Also provided herein is a method of altering the phenotype of a tumor-associated macrophage in a cancer cell, comprising contacting the anticancer agent of the any of the foregoing nanoparticles that comprise an anticancer agent, with the cancer cell.

In some embodiments, the altering comprises converting an M2 phenotype to an M1 phenotype.

Also provided herein is a method of reducing the toxicity, side effects, or both of a chemotherapeutic agent in a patient, comprising administering a therapeutically effective amount of any of the foregoing nanoparticles that comprise a therapeutic agent, or the foregoing pharmaceutical composition, to the patient.

In some embodiments, the chemotherapeutic agent is administered systemically, and comprises a TLR7/8 inhibitor. In some embodiments, the TLR7/8 inhibitor comprises resiquimod (R848).

### Definitions

As used herein, the terms “about” and “approximately” are used interchangeably, and when used to refer to modify a numerical value, encompass a range of uncertainty of the numerical value of from 0% to 10% of the numerical value.

As used herein, the singular forms “a,” “an,” and “the” include plural referents unless the context clearly dictates otherwise.

As used herein, the term “derived from” refers to a compound or moiety that is structurally identical in most respects to the compound to which it refers. In some embodiments, the compound that the moiety is derived from was used as a reagent or intermediate in the synthesis of the compound that is substituted with the moiety. In some embodiments, the moiety , only differs structurally from the compound it is derived from at the portion of the moiety that links to the remainder of the molecule that the moiety substitutes. As used herein, a “derivative” of a particular compound or moiety encompasses compounds and moieties that are derived from the particular compound. For example, 2-hydroxypropyl-α-cyclodextrin is derived from α-cyclodextrin.

As used herein, the term “host macrocycle” refers to any compound or chemical group that is a cyclic group comprising a minimum of 12 ring members (e.g., 12 or more contiguous atoms that form a ring), wherein the cyclic group is capable of binding a compound (e.g., a therapeutic agent, e.g., an anticancer agent) by means of intermolecular forces that, under certain conditions, last greater than 1 second (e.g., greater than 2 seconds, 4 seconds, 10 seconds, 60 seconds, 1 minute, 2 minutes, 5 minutes, 20 minutes, 30 minutes, 1 hour, 2 hours, 4 hours, 1 day, 3 days, 1 week, 2 weeks, 1 month, 2 months, 6 months, 1 year, 2 years, 5 years, or 10 years). Example classes of host macrocycles include cyclodextrins, pillar[n]arenes, calix[n]arenes, and cucurbit[n]urils. Included in each of the foregoing classes are host macrocycles derived from any members of that class through, for example, chemical derivatization. For example, “cyclodextrin” encompasses α-cyclodextrin, β-cyclodextrin, and γ-cyclodextrin, as well as any chemically derivatized versions of the same including, but not limited to, 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, or an aminated cyclodextrin.

As used herein, the term “patient,” refers to any animal, including mammals (e.g. domesticated mammals). Example patients include, but are not limited to, mice, rats, rabbits, dogs, cats, swine, cattle, sheep, horses, primates, and humans.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1B are graphic representations of the tumor microenvironment (FIG. 1A) and the conversion of M2 macrophages to M1 macrophages (FIG. 1B).

FIGS. 2A-2B are a bar graph of gene expression level of M2 and M1 macrophages (FIG. 2A) and a fluorescence image of cell shape of M2 and M1 macrophage polarization states (FIG. 2B).

FIGS. 3A and 3B are an image of an M1 macrophage and automated segmentation showing features of the macrophage (FIG. 3A), and a morphological phenotyping of M1 and M2 macrophages by random forest assignment (FIG. 3B).

FIGS. 4A-4C are chemical structures of tyrosine kinase inhibitors (FIG. 4A), colony-stimulating factor 1 receptor inhibitors (FIG. 4B), and toll-like receptor agonists (FIG. 4C).

FIGS. 5A-5B are a graph of M1 enrichment for a range of drugs (FIG. 5A), and a plot of M1 enrichment vs. log of concentration for motilomod, GS9620, and R848 (FIG. 5B).

FIG. 6 depicts representative fluorescence microscopy images of M2-like macrophage derivation and subsequent conversion to an M1 macrophage.

FIG. 7 is a schematic of cyclodextrin nanoparticle (CDNP) preparation.

FIGS. 8A-8C are a bar graph of CDNP diameter vs. concentration of cyclodextrin used in nanoparticle formation (FIG. 8A), a scanning electron microscopy image of a CDNP (FIG. 8B), and plot of drug loading vs. the molar ratio of R848 to cyclodextrin (FIG. 8C).

FIG. 9 is a plot showing the half-life of a CDNP in tumor-bearing C57BL/6 mice.

FIG. 10 is a fluorescence reflectance image of CDNP-VT680 accumulation in mouse tumors and representative organs at 24 hours following administration of the CDNP-VT680.

FIG. 11 is a bar graph of CDNP-VT680 distribution in various tissue and organs.

FIGS. 12A-12D are confocal fluorescence microscopy images of CDNP-VT680 60 minutes following administration (FIGS. 12A-12B) and 24 hours following administration (FIGS. 12C-12D).

FIG. 13 is a diagram depicting intravital imaging of PacificBlue-ferumoxytol labeled macrophages.

FIG. 14 depicts representative high magnification confocal fluorescence microscopy images of TAMs within tumors 24 hours following administration of CDNP, R844, and CDNP-R848.

FIG. 15 is a scatter dot plot of quantified IL12 expression for CDNP, R848, and CDNP-R848.

FIGS. 16A-16E are a scatter dot plot of tumor area vs. a vehicle, a CDNP, R848, and CDNP-R848 (FIG. 16A), a plot of percent survival vs, time for a vehicle, a CDNP, R848, and CDNP-R848 (FIG. 16B), macroscopic images of mouse tumors at day 8 after treatment with a vehicle, a CDNP, R848, and CDNP-R848 (FIG. 16C), a plot of tumor area vs. time for mice treated with R848 and CDNP-R848 (FIG. 16D), and a waterfall plot of change in tumor area for various treatments at 8 days following each treatment (FIG. 16E).

FIGS. 17A-17C are fluorescence microscopy images of M0 macrophages (FIG. 17A), M2 macrophages (FIG. 17B), and M1 macrophages (FIG. 17C).

FIGS. 18A-18B are a bar graph of IL12 expression for M1 and M2 macrophages and at various concentrations of R848 (FIG. 18A), and a plot of IL12 expression vs. log of R848 concentration (FIG. 18B).

FIGS. 19A-19B are a bar graph of TLR7 expression for M1 and M2 macrophages and at various concentrations of R848 (FIG. 19A), and a plot of TLR7 expression vs. log of R848 concentration (FIG. 19B).

FIGS. 20A-20B are bar graph of gene expression of M1 and M2 macrophages (FIG. 20A), and a correlation between biologically relevant transcriptional markers of M1 and M2 likeness and corresponding gene weights (FIG. 20B).

FIGS. 21A-21B are a bar graph of transcriptional M1-likeness induced by a series of drugs (FIG. 21A) and a plot of M1-likeness vs. M1 enrichment (FIG. 21B).

FIGS. 22A-22B are schematic representations of nanoparticle monomers (FIG. 22A) and the structure/composition of the nanoparticles that are formed from the monomers (FIG. 22B).

FIGS. 23A-23B is a bar graph of CDNP size vs. concentration of CD used in the preparation of the CDNPs (FIG. 23A), and a scatter dot plot of normalized uptake of various nanoparticles into RAW 264.7 cells (FIG. 23B).

FIGS. 24A-24C depict a schematic representation of competitive binding of phenolphthalein and R848 with a CDNP (FIG. 24A), a plot of absorbance vs. wavelength at different concentrations of CDNP (FIG. 24B), and a bar graph of relative absorbance for nanoparticles having various CD content (FIG. 24C).

FIGS. 25A-25B are a pie chart of distribution of CDNP-VT680 in various immune cells in the tumor (FIG. 25A), and a bar graph of CDNP-VT680 distribution into macrophages in various tissue types (FIG. 25B).

FIGS. 26A-26C are qPCR assessment of transcription expressed as fold change relative to M2 (IL-4 treated) controls (FIG. 26A), a bar graph of M1-likeness as a function of treatment of murine macrophages with R848 and CDNP-R848 (FIG. 26B), and a bar graph of the expression of IL12 by human macrophages treated with CDNP, R848, and CDNP-R848 (FIG. 26C).

FIG. 27 depicts flow cytometry plots of IL12-eYPF in TAMs obtained from mice tumors 24 hours after injection with saline, R848, CDNP, and CDNP-R848.

FIGS. 28A-28B are a plot of cancer cell proliferation over 3 days after treatment with DMSO, R848, CDNP, and CDNP-R848 (FIG. 28A) and a bar graph of cell population after treatment with various concentrations of each treatment (FIG. 28B).

FIGS. 29A-29C depict a set of confocal fluorescence microscopy images of a tumor after treatment with CDNP-R848 (FIG. 29A), a bar graph of tumor mass after treatment with R848, CDNP, and CDNP-R848 (FIG. 29B), and gross imaging of resected tumors after each treatment (FIG. 29C).

FIGS. 30A-30B are a plot of tumor area vs. time for a vehicle, R848, CDNP, and CDNP-R848 (FIG. 30A) and a plot of subject percent survival for each treatment (FIG. 30B).

FIG. 31 is a set of photographic images of cancerous mice 7 days after treatment with a vehicle, CDNP, R848, CDNP-R848, aPD-1, and aPD-1+CDNP-R848,

FIGS. 32A-32D are plots of tumor area vs. time for CDNP treatments (FIG. 32A), CDNP-R848 treatments (FIG. 32B), aPD-1 treatments (FIG. 32C), and aPD-1+CDNP-R848 (FIG. 32D).

FIG. 33 is a bar graph showing the ratio of M2:M01:M0 macrophages in a series of cancers.

FIG. 34 shows a fluorescence reflectance image of CDNP-VT680 accumulation in the lungs and liver at 1, 4, and 24 hours following administration of the CDNP-VT680.

FIG. 35 shows a bar graph of CDNP-VT680 distribution in various tissue and organs at 1, 2, and 24 hours following administration of the CDNP-VT680.

FIG. 36 shows confocal fluorescence microscopy images of CDNP-VT680 accumulation in the lung at 1, 4, and 24 hours following administration.

FIG. 37 is confocal fluorescence microscopy images of CDNP-VT680 24 hours following administration.

FIG. 38 shows a bar graph of distribution of CDNP-VT680 in various immune cells in the tumor.

FIGS. 39A-39B show confocal fluorescence microscopy images of CDNP-VT680 and R848-BODIPY TMR X in lung tumors at 24 hours following administration of R848-BODIPY TMR X (FIG. 39A) or CDNP-VT680 and R848-BODIPY (FIG. 39B).

FIG. 40 shows a dot plot of R8480BODIPY-TMR X signal intensity in macrophages in the tumor.

FIG. 41 shows a plot of tumor area vs. time for a vehicle control and CDNP-R848, with or without CD8 depletion or CD8 intact.

FIG. 42 shows a set of photographic images of cancerous mice 6 days after treatment with a vehicle, CDNP, R848, CDNP-R848, aPD-1, and aPD-1+CDNP-R848.

FIGS. 43A-43B show a plot of tumor area vs. time after treatment with a vehicle, CDNP, R848, CDNP-R848, aPD-1, and aPD-1+CDNP-R848. (FIG. 43A) and a plot of subject percent survival for each treatment (FIG. 43B).

### DETAILED DESCRIPTION

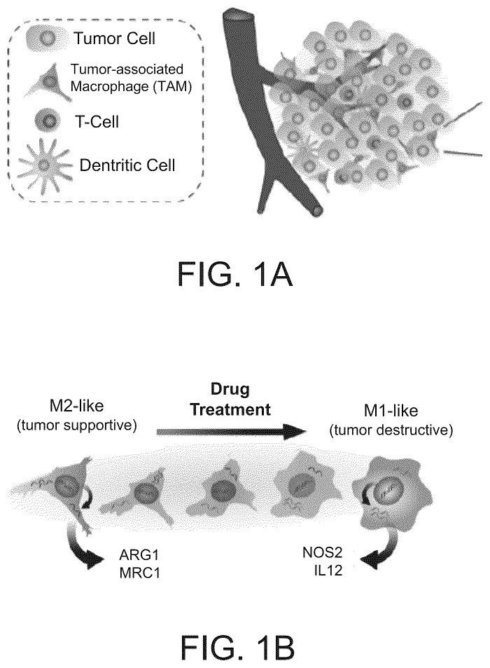

Tumor-associated macrophages (TAMs) play roles in tumor metastasis and resistance to therapeutic drugs. TAMs can assume opposing phenotypes that can be either tumorigenic (e.g., M2-like cells) or tumoricidal (e.g., M1-like cells). In some tumors, the tumorigenic M2 phenotype prevails. TAMs having the M2 phenotype can accelerate the progression of untreated tumors and adversely influence the effectiveness and/or efficacy of anticancer drugs. Small molecules that inhibit receptors, tyrosine kinases, and/or other transduction pathways in TAMs, and that convert (i.e., re-educate) M2 TAMs into M1 TAMs, have been developed. Such drugs are administered systemically and as such are not delivered to the tumor selectively, leading to side effects. Disclosed herein are nanoparticles that, in some embodiments, include a therapeutic agent (e.g., an anticancer agent, e.g., an anticancer agent that converts M2 TAMs to M1 TAMs, e.g., resiquimod (R848)). In some embodiments, when administered to a patient, the therapeutic nanoparticles provided herein can release the therapeutic agent into the tumor microenvironment (FIG. 1A), where the therapeutic agent can be subsequently taken up into TAMs. In some embodiments, the release of the therapeutic agent induces M2 to M1 phenotype conversion in the TAM (FIG. 1B). The nanoparticles provided herein can include one or more of the following characteristics: safe, biocompatible, high loading capacities, biodegradable following release of their therapeutic payload, and high affinity for TAMs. In some embodiments, the nanoparticles can be used to treat micro-metastases and surgically inaccessible tumors, for example, where intratumoral injections are difficult or not feasible.

### Nanoparticles

The nanoparticles disclosed herein comprise at least two host macrocycles, wherein the at least two host macrocycles are covalently crosslinked by a linker.

In some embodiments, at least one of the at least two host macrocycles (e.g., at least two of the at least two host macrocycles) is selected from a cyclodextrin (CD), a pillar[n]arene, a calix[n]arene, or a cucurbit[n]uril. In some embodiments, at least one of the host macrocycles is a cyclodextrin. Examples of cyclodextrins include α-cyclodextrin, β-cyclodextrin, γ-cyclodextrin, 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, or an aminated cyclodextrin. In some embodiments, the cyclodextrin is β-cyclodextrin.

In some embodiments, the linker is formed by means of, for example, metal-catalyzed cross-coupling reactions, condensation reactions, addition reactions, or free radical polymerizations. In some embodiments, the linker crosslinks two host macrocycles through a reactive group (e.g., a hydroxyl, amino, amino, sulfoxyl, sulfhydryl, haloacyl, or carboxyl group) on each host macrocycle.

In some embodiments, the linker comprises chemical groups derived from natural and/or unnatural amino acids (e.g., lysine (e.g., L-lysine or D-lysine), arginine, histidine, aspartic acid, glutamic acid, serine, threonine, asparagine, glutamine, cysteine, glycine, tyrosine, tryptophan), succinimides (e.g., N-hydroxysuccinimide), alkylene diamines, epoxides, or epichlorohydrin.

In some embodiments, the linker comprises a moiety of Formula (I):

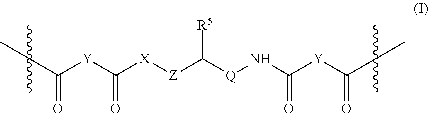

wherein:

Q is selected from a bond or methylene;

X is selected from O, S, and NR¹;

each Y is independently selected from C₁₋₁₀ alkylene optionally substituted with one or more R²;

Z is selected from A-B, wherein A is selected from a bond and C₁₋₁₀ alkylene, and B is selected from C₁₋₁₀ arylene, 3-10 membered heteroarylene, 3-10 membered heterocycloalkyl, and C₃₋₁₀ cycloalkyl;

wherein A is optionally substituted with one or more R³, and B is optionally substituted with one or more R⁴;

R¹ is selected from H and C₁₋₃ alkyl;

each R² is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

each R³ is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

each R⁴ is independently selected from C₁₋₁₀ arylene, 3-10 membered heteroarylene, 3-10 membered heterocycloalkyl, C₃₋₁₀ cycloalkyl, hydroxy, halo, CN, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and

R⁵ is selected from H, C₁-C₆ alkyl, CO₂H, C₁₋₁₀ arylene, 3-10 membered heteroarylene, 3-10 membered heterocycloalkyl, C₃₋₁₀ cycloalkyl, hydroxy, halo, CN, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl ; and

wherein each [symbol] through a bond indicates a point of attachment to a host macrocycle or an additional moiety that attaches to the host macrocycle.

In some embodiments, R⁵ is CO₂H. In some embodiments, Q is a bond. In some embodiments, each Y is ethylene. In some embodiments, X is NH. In some embodiments, Z is n-butylene.

In some embodiments, the at least two host macrocycles comprise less than 1×10⁹ host macrocycles (e.g., less than 1×10⁸, less than 1×10⁷, less than 5×10⁶, less than 1×10⁶, less than 1×10⁵, less than 1×10⁴, less than 5000, less than 2500, less than 1000, less than 500, or less than 100). For example, the nanoparticles comprise from 2 to 1×10⁹ host macrocycles, from 2 to 1×10⁸ host macrocycles, from 2 to 1×10⁷ host macrocycles, from 2 to 5×10⁶ host macrocycles, from 2 to 1×10⁶ host macrocycles, from 2 to 1×10⁵ host macrocycles, from 2 to 1×10⁴ host macrocycles, from 2 to 5000 host macrocycles, from 2 to 2500 host macrocycles, from 2 to 1000 host macrocycles, from 2 to 500 host macrocycles, or from 2 to 100 host macrocycles.

In some embodiments, the nanoparticle comprises at least one polymer. In some embodiments, the polymer is linear. In some embodiments, the polymer is branched. Example polymers include a dextran derivative, a hyaluronic acid derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative, a oligo(poly(ethylene glyco)fumarate) derivative, a poly(vinyl alcohol) derivative, and a poly(vinyl acid) derivative.

In some embodiments, the nanoparticle comprises at least one therapeutic agent. In some embodiments, the at least one therapeutic agent forms a host-guest complex with at least one of the host macrocycles. In some embodiments, the at least one therapeutic agent is covalently bonded with at least one of the host macrocycles. In some embodiments, the at least one therapeutic agent comprises an anticancer or immunomodulating agent. In some embodiments, the at least one therapeutic agent comprises an anticancer agent. In some embodiments, the anticancer agent is a toll-like receptor (TLR) agonist. In some embodiments, the anticancer agent is a TLR3, TLR4, TLR 7/8, or TLR9 agonist. For example, the anticancer agent is a TLR 7/8 agonist. In some embodiments, the TLR 7/8 agonist is imiquimod, gardiquimod, resiquimod (R848), motolimod, or GS9620. For example the TLR 7/8 agonsit is resiquimod (R848), in some embodiments, one or more of the at least one therapeutic agents is selected from the group consisting of: GW2580, CEP32496, BL1945, OSI930, PLX3397, dasatinib, sunitinib, ABT869, imatinib, foretinib, XL228, gefitinib, PD0325901, trametinib, bentamapimod, dabrafenib, vemurafinib, crizotinib, UNC2025, indoximod, celecoxib, rapamycin, NIK12192, trichostatin A, IBET151, TMP195, BYL719, GDC0941, BKM120, imiquimod, gardiquimod, resiquimod (R848), motolimod, and GS9620. In some embodiments, one or more of the at least one therapeutic agents is a compound selected from the group consisting of: imiquimod, indoximod, gardiquimod, motolimod, or resiquimod (R848). For example, one or more of the at least one therapeutic agents is resiquimod (R848). In some embodiments, one or more of the at least one therapeutic agents comprises a 1H-imidazo[4,5-c]quinoline. In some embodiments, one or more of the at least one therapeutic agents comprises a 4-amino-1H-imidazo[4,5-c]quinoline. In some embodiments, the therapeutic agent is a TKi, CSF1R, or HDAC inhibitor.

In some embodiments, the nanoparticle comprises two or more therapeutic agents. In some embodiments, one of the two or more therapeutic agents improves the efficacy of one or more of the other therapeutic agents (e.g., is synergistic with one or more of the other therapeutic agents).

In some embodiments, nanoparticle further comprises an imaging agent. In some embodiments, the imaging agent comprises a magnetic resonance imaging (MRI) agent, a positron emission tomography (PET) agent, a single-photon emission computed tomography (SPECT) agent, or a near-infrared fluorophore. In some embodiments, the imaging agent comprises a near-infrared fluorophore. In some embodiments, the near-infrared fluorophore is selected from the group consisting of Vivo Tag 680-XL, ZW800-1C, ZW800-1, ZW800-3C, ZW700-1, indocyanine green (ICG), Cy5, Cy5.5, Cy7, Cy7.5, IRDye800-CW (CW800), BODIPY 630, and ZWCC.

In some embodiments, the one or more therapeutic agents are conjugated with a fluorescent dye. In certain instances, conjugating a fluorescent dye to the therapeutic agent enables tracking (e.g., imaging) of the therapeutic agent in vivo. In some embodiments, the fluorescent dye includes a xanthene derivative (e,g., fluorescein, rhodamine, Oregon green, eosin, or Texas red), cyanine derivative (e.g., cyanine, indocarbocyanine, oxacarbocyanine, thiacarbocyanine, or merocyanine), squaraine derivative or ring-substituted squaraine (e.g., seta, setau, and square dyes), naphthalene derivative (e.g., dansyl or prodan derivatives), coumarin derivative, oxadiazole derivative (e.g., pyridyloxazole, nitrobenzoxadiazole, or benzoxadiazole), anthracene derivative (e.g., anthraquinones, including DRAQ5, DRAQ7, or CyTRAK orange), pyrene derivative (e.g., cascade blue), oxazine derivative (e.g., nile red, nile blue, cresyl violet, or oxazine 170), acridine derivative (e.g., proflavin, acridine orange, or acridine yellow), arylmethine derivative (e.g., auramine, crystal violet, or malachite green), tetrapyrrole derivative (e.g., porphin, phthalocyanine, or bilirubin), ZW800 (e.g., ZW800-1C, ZW800-1, or ZW800-3C), ZW700-1, indocyanine green (ICG), Cy5, Cy5.5, Cy7, Cy7.5, IRDye800-CW (CW800), or ZWCC. In some embodiments, the fluorescent dye is a xanthene derivative (e.g., fluorescein, rhodamine, Oregon green, eosin, or Texas red), cyanine derivative cyanine, indocarbocyanine, oxacarbocyanine, thiacarbocyanine, or merocyanine), squaraine derivative or ring-substituted squaraine (e.g., seta, setae, and square dyes), naphthalene derivative (e.g., dansyl or prolan derivatives), coumarin derivative, oxadiazole derivative (e.g., pyridyloxazole, nitrobenzoxadiazole, or benzoxadiazole), anthracene derivative (e.g., anthraquinones, including DRAQ5, DRAQ7, or CyTRAK, orange), pyrene derivative (e.g., cascade blue), oxazine derivative (e.g., nile red, nile blue, cresyl violet, or oxazine 170), acridine derivative (e.g., proflavin, acridine orange, or acridine yellow), arylmethine derivative (e.g., auramine, crystal violet, or malachite green), tetrapyrrole derivative (e.g., porphin, phthalocyanine, or bilirubin), ZW800 (e.g., ZW800-1C, ZW800-1, or ZW800-3C), ZW700-1, indocyanine green (ICG), Cy5, Cy7, Cy7.5, IRDye800-CW (CW800), or ZWCC. In some embodiments, the at least one therapeutic agent is conjugated with adamantane.

In some embodiments, the stoichiometric ratio of the cyclodextrin to the therapeutic agent is from about 100:1 to about 1:100 (e.g., from about 100:1 to about 1:100, from about 100:1 to about 1:1, from about 50:1 to about 1:1, from about 20:1 to about 1:1, from about 10:1 to about 1:1, from about 5:1 to about 1:1, from about 2:1 to about 1:1, from about 1:1 to about 100:1, from about 1:1 to about 50:1, from about 1:1 to about 20:1, from about 1:1 to about 10:1, from about 1:1 to about 5:1, from about 1:1 to about 2:1, from about 50:1 to about 1:50, from about 20:1 to about 1:20, from about 10:1 to about 1:10, from about 2:1 to about 1:2, about 1.1:1, or about 1:1. In some embodiments, the stoichiometric ratio of the cyclodextrin to the therapeutic agent is about 1.1:1.

In some embodiments, the equilibrium binding constant (K_{D}) of the at least one therapeutic agent to the host macrocycle is from about 1×10⁻¹² M to about 0.1 M (e.g., from about 1×10⁻¹¹ M to about 0.1 M, from about 1×10⁻¹⁰ M to about 0.1 M, from about 1×10⁻⁹ M to about 0.1 M, from about 1×10⁻⁸ M to about 0.1 M, from about 1×10⁻⁷ M to about 0.1 M, from about 1×10⁻⁶ M to about 0.1 M, from about 1×10⁻⁵ M to about 0.1 M, from about 1×10⁻⁴ M to about 0.1 M, from about 1×10⁻³ M to about 0.1 M, from about 1×10⁻² M to about 0.1 M, from about 1 mM to about 10 mM, from about 2 mM to about 8 mM, from about 5 mM to about 8 mM, from about 5.5 mM to about 7.2 mM, or about 0.1 M). In some embodiments, the equilibrium binding constant (K_{D}) of the at least one therapeutic agent to the host macrocycle is from about 5.5 mM to about 7.2 mM (e.g., about 5.5 mM, about 6 mM, about 6.3 mM, about 7 mM, or about 7.2

In some embodiments, the half-life of the therapeutic agent in vivo after release from the nanoparticle is from about 30 to about 120 minutes (e.g., from about 30 to about 90 minutes, from about 40 to about 90 minutes, from about 90 to about 120 minutes, from about 90 to about 100 minutes, from about 45 to about 90 minutes, about 55 minutes, about 60 minutes, about 62 minutes, or about 65 minutes). In some embodiments, the half-life of the therapeutic agent in vivo after release from the nanoparticle is about 62 minutes.

In some embodiments, the nanoparticle has an overall negative charge. In some embodiments, the nanoparticle has a zeta potential of from about −5 mV to about −50 mV (e.g., from about −10 mV to about −50 mV, from about −15 mV to about −50 mV, from about −20 mV to about −50 mV, from about −30 mV to about −50 mV, from about −40 mV to about −50 mV, from about −5 mV to about −40 mV, from about −5 mV to about −30 mV, from about −-5 mV to about −20 mV, from about −5 mV to about −10 mV, or about −10 mV). In some embodiments, the nanoparticle has a zeta potential of about −10 mV.

In some embodiments, the average molecular weight of the nanoparticle is from about 1,500 g/mol to about 5×10¹¹ g/mol (e.g., from about 1,500 g/mol to about 5×10¹⁰ g/mol, from about 1,500 g/mol to about 5×10⁹ g/mol, from about 1,500 g/mol to about 5×10⁸ g/mol, from about 1,500 g/mol to about 5×10⁷ g/mol, from about 1,500 g/mol to about 5×10⁶ g/mol, from about 1,500 g/mol to about 5×10⁵ g/mol, from about 1,500 g/mol to about 5×10⁴ g/mol, from about 1,500 g/mol to about 5×10³ g/mol, from about 5×10³ g/mol to about 5×10¹¹ g/mol, 5×10⁴ g/mol to about 5×10¹¹ g/mol, 5×10⁵ g/mol to about 5×10¹¹ g/mol, 5×10⁶ g/mol to about 5×10¹¹ g/mol, 5×10⁷ g/mol to about 5×10¹¹ g/mol, 5×10⁸ g/mol to about 5×10¹¹ g/mol, 5×10⁹ g/mol to about 5×10¹¹ g/mol, 15×10³ g/mol to about 20×10⁶ g/mol, or about 20×10⁶ g/mol). In some embodiments, the average molecular weight of the nanoparticle is from about 15×10³ g/mol to about 20×10⁶ g/mol. In some embodiments, the average molecular weight of the nanoparticle is about 20×10⁶ g/mol.

In some embodiments, the nanoparticle comprises an average of from about 10 to about 10,000 cyclodextrins (e.g., from about 10 to about 10,000 cyclodextrins, from about 100 to about 10,000 cyclodextrins, from about 1000 to about 10,000 cyclodextrins, from about 2000 to about 10,000 cyclodextrins, from about 5000 to about 10,000 cyclodextrins, from about 8000 to about 10,000 cyclodextrins, from about 100 to about 8,000 cyclodextrins, from about 100 to about 5,000 cyclodextrins, from about 100 to about 2,000 cyclodextrins, from about 100 to about 1,000 cyclodextrins, about 500 cyclodextrins, about 1,000 cyclodextrins, or about 2,000 cyclodextrins. In some embodiments, the nanoparticle comprises an average of from about 100 to about 2,000 cyclodextrins, In some embodiments, the nanoparticle comprises an average of about 1,000 cyclodextrins.

In some embodiments, the average hydrodynamic diameter of the nanoparticle is from about 10 nm to about 1000 nm (e.g, from about 10 nm to about 500 nm, from about 10 nm to about 300 nm, from about 10 nm to about 200 nm, from about 10 nm to about 100 nm, from about 100 nm to about 1000 nm, from about 200 nm to about 1000 nm, from about 500 nm to about 1000 nm, from about 700 nm to about 1000 nm, from about 10 nm to about 70 nm, from about 20 to about 60 nm, about 50 nm, or about 30 nm). In some embodiments, the average hydrodynamic diameter of the nanoparticle is from about 10 nm to about 70 nm. In some embodiments, the average hydrodynamic diameter of the nanoparticle is from about 20 to about 60 nm. In some embodiments, the average hydrodynamic diameter of the nanoparticle is about 50 nm. In some embodiments, the average hydrodynamic diameter of the nanoparticle is about 30 nm.

### Methods of Use

The present application further provides methods of treating a disease or disorder in a patient (e.g., cancer), including administering a therapeutically effective amount of the nanoparticle (or a composition (e.g., a pharmaceutical composition) comprising the nanoparticle) provided herein to the patient. In some embodiments, the nanoparticle comprises one or more therapeutic agents. In such embodiments, for example, a therapeutically effective amount of the nanoparticle can be determined based upon the amount of therapeutic agent to be administered to the patient by the nanoparticle.

In some embodiments, the cancer comprises a tumor-associated macrophage (TAM). In some embodiments, the phenotype of the tumor-associated macrophage is M2. It is understood that, in some embodiments, the M2 tumor-associated macrophage encourages tissue repair and/or deactivates immune system activation in tumors (by, for example, metabolizing arginine to the ornithine, which facilitates the repair or by producing anti-inflammatory cytokines such as IL-10). In some embodiments, the treating further comprises converting (i.e., re-educating) the phenotype of the macrophage from M2 to M1. In some embodiments, the M1 tumor-associated macrophage encourages inflammation and tissue disrepair (by, for example, secreting high levels of IL-12 and low levels of IL-10, and/or by metabolizing arginine to nitric oxide). Not wishing to be bound by theory, it is understood that the phenotype conversion of tumor-associated macrophages from M2 to M1 exerts an anticancer effect by, for example, slowing cancer growth (e.g., reducing the rate of cancer growth, e.g., reducing the rate of formation of cancer cells.), stopping cancer growth, or killing cancer cells.

In some embodiments, the cancer is selected from the group consisting of Ewing sarcoma, osteosarcoma, glioblastoma, meningioma, oligodendrial cancer, melanoma metastasis, melanoma primary, breast cancer, gastric cancer, germ cell tumors, astrocytoma, ovarian cancer, lung large cell carcinoma, lung adenocarcinoma, lung squamous cell carcinoma, colon cancer, head and neck cancer, bladder cancer, thyroid cancer, liver cancer, pancreas cancer, kidney cancer, cervical cancer, testicular cancer, prostate cancer, and bone cancer. In some embodiments, the cancer is metastatic.

In some embodiments, the nanoparticle comprising the anticancer agent kills the cancer faster than the anticancer agent alone. In some embodiments, the nanoparticle comprising the anticancer agent kills more cancer cells than the anticancer agent alone after 6 hours, 12 hours, 1 day, 2 days, 4 days, 6 days. 8 days, 2 weeks, 1 month, 2 months, 4 months, 6 months, or 1 year following administration of one or more doses of the anticancer agent.

In some embodiments, the uptake of the nanoparticle is higher into the tumor and/or into tumor associated macrophages than into any other organ or tissue type in the subject after administration (e.g., muscle, heart, or liver). In some embodiments, less than 50 mol % of the nanoparticle is released prior to uptake of the nanoparticle into tumor macrophage cells (e.g., less than 40 mol %, less than 30 mol %, less than 20 mol %, less than 10 mol %, less than 7 mol %, less than 5 mol %, less than 2 mol %, or less than 1 mol % of the nanoparticle is released prior to uptake of the nanoparticle into tumor macrophage cells). In some embodiments, less than 10 mol % of the nanoparticle is released prior to uptake of the nanoparticle into tumor macrophage cells. In some embodiments, less than 5 mol % of the nanoparticle is released prior to uptake of the nanoparticle into tumor macrophage cells. In some embodiments, less than 1 mol % of the nanoparticle is released prior to uptake of the nanoparticle into tumor macrophage cells.

In some embodiments, the method further comprises administering an additional therapeutic agent in combination with a nanoparticle that comprises a therapeutic agent that improves the efficacy of the therapeutic agent (e.g., is synergistic with the therapeutic agent). In some embodiments, the additional therapeutic agent, in combination with the nanoparticle comprising the therapeutic agent, kills the cancer faster than the nanoparticle comprising the therapeutic agent alone (i.e., without an additional therapeutic agent). In some embodiments, the additional therapeutic agent is a PD-1 antibody, a CTLA-4 antibody, a PD-L1 antibody, an inhibitor, a CSF-1R inhibitor, kinase inhibitor, an HDAC inhibitor, a PI3K inhibitor, a MerTK inhibitor, or an Ax1 inhibitor. In some embodiments, the additional therapeutic agent is a PD-1 antibody. In some embodiments, the PD-1 antibody is selected from the group consisting of: nivolumab, pembrolizumab, pidilizumab, BMS-936559, atezolizumab, and avelumab. In some embodiments, the additional therapeutic agent is selected from afatinib, AG 879, alectinib (Alecensa), altiratinib, apatinib (Tykerb), ARQ-087, ARRY-112, ARRY-523, ARRY-651, AUY-922, AXD7451, AZ-23, AZ623, AZ64, AZD4547, AZD6918, AZD7451, BGJ398, binimetinib, BLU6864, BLU9931, brivatinib, cabozantinib, CEP-751 and CEP-701 (lestaurtinib), cetuximab (Erbitux), CH5183284, crizotinib (Xalkori), CT327, dabrafenib (Tafinlar), danusertib, DCC-2036 (rebastinib), DCC-2157, dovitinib, DS-6051, encorafenib, erdafitinib, erlotinib, EWMD-2076, gefitinib (Iressa), GNF-4256, GNF-5837, Gö 6976, GTx-186, GW441756, imatinib (Gleevec), K252a, Iapatinib, lenvatinib (Lenvima), Loxo-101, Loxo-195 (ARRY-656), lucitanib, LY2874455, MGCD516 (sitravatinib), motesanib, nilotinib (Tasigna), nintedanib, NVP-AST487, ONO-5390556, orantinib (TSU-68, panitumumab (Vectibix), pazopanib (Votrient), PD089828, PD166866, PD173074, pertuzumab (Perjeta), PF-477736, PHA-739358 (danusertib), PHA-848125AC (Milciclib), PLX7486, ponatinib (AP-24534), PZ-1, quercetin, regorafenib (Stivarga), RPI-1, ruxolitinib, RXDX101 (Entrectinib), RXDX105, semaxanib (SU5416), sorafenib, SPP86, SSR128129E, SU4984, SU5402, SU6668, SUN11602, Sunitinib, TAS120, TG101209, TPX-0005, trastuzumab, TSR-011, vandetanib (Caprelsa), vatalanib, VSR-902A, and XL-184 (cabozantinib).

In some embodiments, the method further comprises treating the patient with radiation, chemotherapy, antibody checkpoint therapy, immunotherapy, or any combination thereof.

The present application further provides methods of imaging a tissue in a subject, including administering the nanoparticle provided herein to the patient. In some embodiments, the tissue includes cancer cells. In some embodiments, the tissue includes kidney tissue, bladder tissue, or both.

In some embodiments, the patient is a mammal (e.g., a human or a domesticated mammal).

The present application further provides methods of altering the phenotype of a tumor-associated macrophage in a cancer cell, comprising contacting a therapeutic agent (e.g., an anticancer agent) of a nanoparticle disclosed herein with the cancer cell. In some embodiments, the altering comprises converting an M2 phenotype to an M1 phenotype.

The present application further provides methods of reducing the toxicity, side effects, or both of a chemotherapeutic agent in a patient, comprising administering a therapeutically effective amount of a nanoparticle comprising the chemotherapeutic agent as disclosed herein to the patient. In some embodiments, the nanoparticle comprising the chemotherapeutic agent is administered systemically (e.g., intraperitoneally, intravenously, intraarterially), and comprises a TLR7/8 inhibitor (e.g., resiquimod).

### Methods of Preparation

The nanoparticles disclosed herein (e.g., nanoparticles comprising cyclodextrins) may be formed by, for example, reacting a 6′-hydroxyl group of a cyclodextrin with N-hydroxysuccinimide (NHS) to form an ester bond to result in a succinyl-β-cyclodextrin. Subsequent amide bond formation between a free carboxyl group on a succinyl-β-cyclodextrin and a free carboxyl group on another succinyl-β-cyclodextrin with two amino groups of L-lysine results in a crosslinked nanoparticle (Scheme 1). In some embodiments, a molar ratio of 1:2 lysine to succinyl moieties is used in the crosslinking step. In some embodiments, a solution comprising from about 0.5% to about 109 wt/vol is used in the crosslinking step (e.g., from about 0.5% to about 8% wt/vol, from about 0.5% to about 5% wt/vol, from about 0.5% to about 3% wt/vol, from about 0.5% to about 2% wt/vol, from about 2% to about 10% wt/vol, from about 3% to about 10% wt/vol, from about 5% to about 10% wt/vol, about 1.25% wt/vol, about 2.5% wt/vol, about 3.3% wt/vol, or about 5% wt/vol). In some embodiments, a solution comprising about 3.3% wt/vol is used in the crosslinking step.

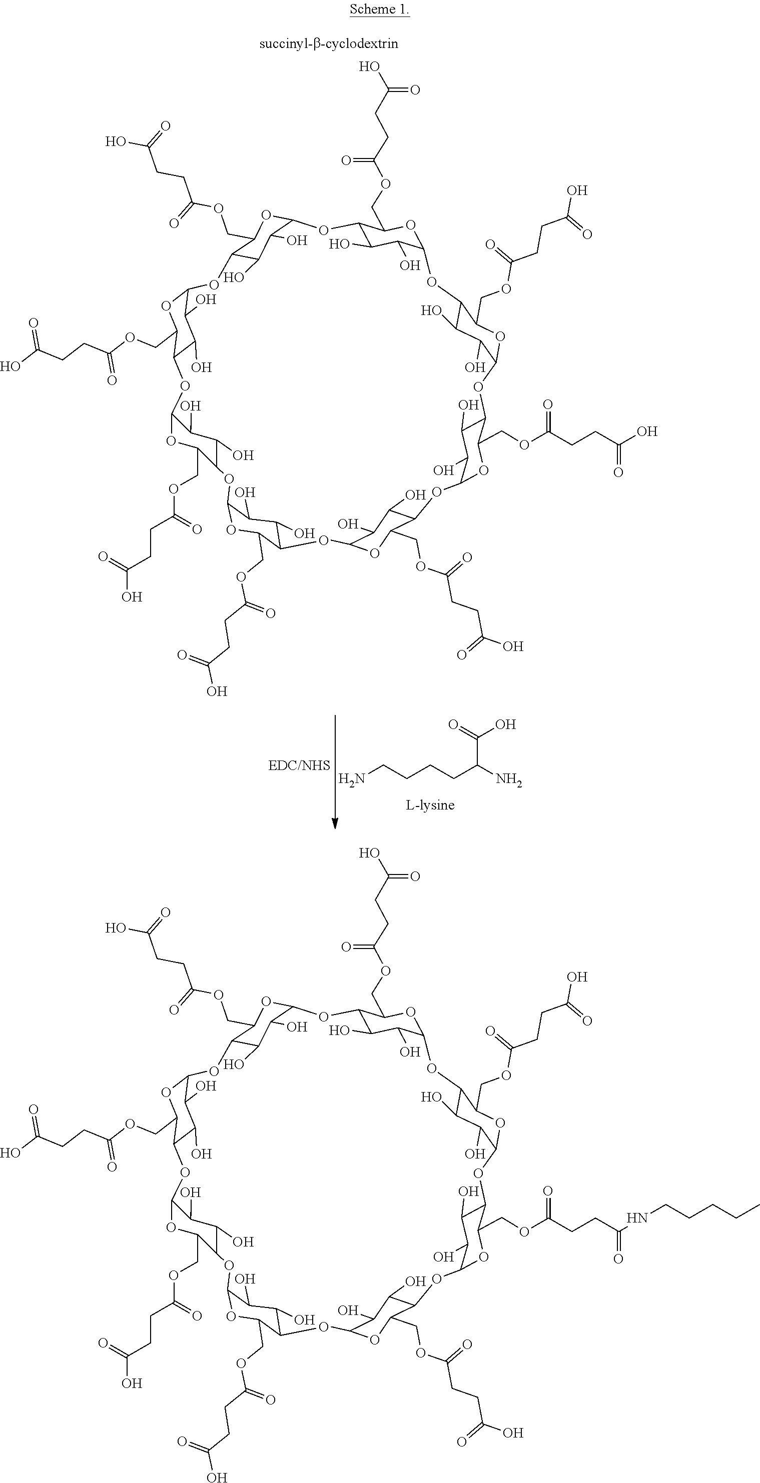

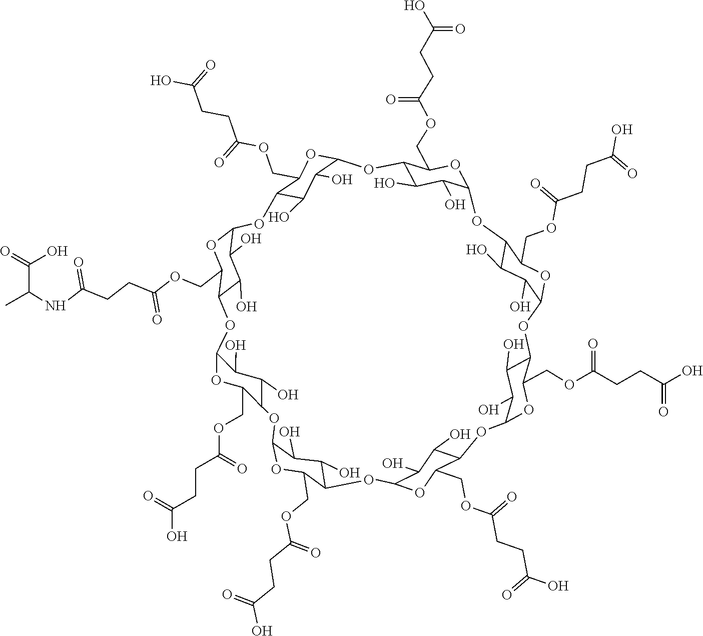 [SMILES: CC(NC(=O)CCC(=O)OCC1OC2OC3C(COC(=O)CCC(=O)O)OC(OC4C(COC(=O)CCC(=O)O)OC(OC5C(COC(=O)CCC(=O)O)OC(OC6C(COC(=O)CCC(=O)O)OC(OC7C(COC(=O)CCC(=O)O)OC(OC8C(COC(=O)CCC(=O)O)OC(OC9C(COC(=O)CCC(=O)O)OC(OC1C(O)C2O)C(O)C9O)C(O)C8O)C(O)C7O)C(O)C6O)C(O)C5O)C(O)C4O)C(O)C3O)C(=O)O]

### Pharmaceutical Compositions and Formulations

When employed as pharmaceuticals, the nanoparticles (e.g., nanoparticles comprising one or more therapeutic agents) provided herein can be administered via various routes (e.g., intravenous, intraarterial, intratumoral, intranasal, subcutaneous, intradermal, intraperitoneal, or oral administration) in the form of pharmaceutical compositions. These compositions can be prepared as described herein or elsewhere, and can be administered by a variety of routes, depending upon whether local or systemic treatment is desired and upon the area to be treated. In some embodiments, the administration is parenteral. Parenteral administration includes, for example, intravenous, intraarterial, subcutaneous, intraperitoneal intramuscular or injection or infusion; or intracranial administration, (e.g., intrathecal or intraventricular, administration). Parenteral administration can be in the form of a single bolus dose, or may be, for example, by a continuous perfusion pump. In some embodiments, the compounds, salts, and pharmaceutical compositions provided herein are suitable for parenteral administration. In some embodiments, the nanoparticles provided herein are suitable for intravenous administration. Conventional pharmaceutical carriers, aqueous, powder or oily bases, thickeners and the like may be necessary or desirable.

Also provided are pharmaceutical compositions which contain, as the active ingredient, a nanoparticle provided herein (e.g., a nanoparticle comprising a therapeutic agent), in combination with one or more pharmaceutically acceptable carriers (e.g., excipients). In making the compositions provided herein, the active ingredient is typically, mixed with an excipient, diluted by an excipient or enclosed within such a carrier in the form of, for example, a capsule, tablet, or other container. When the excipient serves as a diluent, it can be a solid, semi-solid, or liquid material, which acts as a vehicle, carrier or medium for the active ingredient. Thus, the compositions can be in the form of tablets, pills, powders, suspensions, emulsions, solutions, syrups, aerosols (as a solid or in a liquid medium), soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The nanoparticles (e.g., nanoparticles comprising one or more therapeutic agents) can be effective over a wide dosage range and are generally administered in a pharmaceutically effective amount. It will be understood, however, that the amount of the nanoparticle (e.g., nanoparticles comprising one or more therapeutic agents) actually administered will usually be determined by a physician, according to the relevant circumstances, including the condition to be treated, the chosen route of administration, the actual compound administered, the age, weight, and response of the individual subject, the severity of the subject's symptoms, and the like.

### EXAMPLES

The following examples are offered for illustrative purposes, and are not intended to limit the invention.

### Materials

Unless otherwise indicated, solvents and reagents were purchased from Sigma-Aldrich and used without further purification. Water used for all experiments was purified using a MilliQ filtration system (Waters). All pharmacological drugs were purchased from commercial suppliers (Selleckchem, MedchemExpress, InvivoGen, or LC Laboratories). The Rat IgG2a kappa anti-mouse PD1 29F.1A12 clone was provided by Gordon Freeman (DFCI). Ferumoxytol (AMAG Pharmaceuticals) and amino-dextran (500kDa, Thermo Fisher Scientific) were used for intravital imaging, and were fluorescently labeled by Pacific Blue (label concentration: 40.1±2.6 nM m⁻¹ Dextran, 1.79 mg injected).

### Cell Models

Cells were maintained in the indicated medium at 37° C. and 5% CO₂ and screened regularly for mycoplasma. RAW 264.7 cells were sourced from ATCC and maintained in Dutbecco's Modified Eagles Medium supplemented with 10% heat inactivated fetal calf serum (Atlanta Biologicals), 100 IU penicillin (Invitrogen), and 100 μg mL⁻¹ streptomycin (Invitrogen), and 200 mM L-glutamine. The MC38 mouse colon adenocarcinoma cell lines were provided by Mark Smyth (QIMR Berghofer Medical Research Institute) with stable transfection of the H2B-Apple reporter to yield a MC38-H2B-mApple cell line employed in intravital microscopy studies. Murine bone marrow-derived macrophages (BMDMs) were isolated and derived by adaptation of published procedures known to those of skill in the art. Briefly, bone marrow was extracted from the surgically resected femur and tibia of naive C57BL/6 mice, dissociated and passed through a 40 μm strainer, and red blood cells lysed by ammonium chloride (StemCell Tech). Resultant bone marrow cells were plated in either 24-well (Corning 3527, for PCR analysis) or optical-bottom 384-well plates (Thermo Fisher 142761, for image analysis) at 1×10⁶ cells mL⁻¹ in Iscove's Modified Dulbecco's Medium supplemented with 10% heat inactivated fetal calf serum, 100 IU penicillin, 100 μg ml⁻¹ streptomycin (Invitrogen) and 10 ng mL⁻¹ recombinant murine M-CSF (PeproTech, 315-02); media was replenished every two days. Human macrophages were derived from peripheral blood mononuclear cells isolated using Ficoll-Paque PLUS (GE Healthcare) gradient separation and derived in the presence of 50 ng mL⁻¹ recombinant human M-CSF (PeproTech, 300-25). Cell proliferation was assessed by PrestoBlue (Fisher) following manufacturer's protocols.

### Animal Models

Animal research was conducted in compliance with the Institutional Animal Care and Use Committees at Massachusetts General Hospital (MGH). Unless otherwise stated, experiments were performed using female C57BL/6 that were 6- to 8-week old at the start of the experiment, and animals were sourced from The Jackson Laboratory.

### In Vitro Phenotyping

For morphological analysis, media was replenished with M-CSF-tree media on day 7 followed by drug dosing (Table 1).

TABLE 1

| Module | Description |
|---|---|
| GaussianFilter | To smooth nuclear staining for improved |
| | identification of individual nuclei (primary object). |
| | Sigma: 2 |
| ImageMath | To normalize wheat germ agglutinin signal |
| | from improved segmentation of cell membrane |
| | staining (secondary object). |
| | Operation: Log transform (base 2) |
| | Multiply the first image by: 1.0 |
| | Raise the power of the result by 0.5 |
| | Multiply ths result by: 1.0 |
| | Add to result: 0 |
| | Set values less then 0 equal to 0: Yes |
| | Set values greater than 1 equal to 1? Yes |
| | Ignore the image mask? No |
| IdentifyPrimaryObjects | To identify individual cell nuclei, stained by DAPI. |
| | Object: Nuclei |
| | Typical diameter (Min, Max): 4, 20 |
| | Discard objects outside the diameter range? Yes |
| | Discard objects touching the border of the image? Yes |
| | Threshold strategy: Adaptive |
| | Thresholding method: minimum cross entropy |
| | Threshold smoothing scale: 0 |
| | Threshold correction factor: 1.5 |
| | Lower and upper bounds on threshold: 0.0005, 1.0 |
| | Method to distinguish clumped objects: Shape |
| | Method to draw dividing lines between clumped objects: Intensity |
| | Automatically calculate size of smoothing filter for declumping? Yes |
| | Automatically calculate minimum allowed distance between local maxima? No |
| | Speed up by using lower-resolution images to find local maxima? Yes |
| | Fill holes in identified objects? After both thresholding and declumping |
| | Handling of objects if excessive number of objects identified: Continue |
| IdentifySecondaryObjects | To identify cytoplasmic domain, stained by wheat germ agglutinin, associated |
| | with each nuclei. |
| | Select the input objects: Nuclei |
| | Name the objects to be identified: Cells |
| | Select the method to identify the secondary objects: Watershed - image |
| | Threshold strategy: Global |
| | Thresholding Method: Minimum cross entropy |
| | Threshold smoothing scale: 1.3488 |
| | Threshold correction factor: 2.0 |
| | Lower and upper bounds on threshold: 0.075, 0.9 |
| | Fill holes in identified objects? Yes |
| | Discard secondary objects touching the border of the image? Yes |
| | Retain outlines of the identified secondary objects? Yes |
| MesureObjectSizeShape | All available shape features are examined on a cell-by-cell basis.* |
| ExportToDatabase | Export cell shape features for further analysis as described in The Methods. |

*All shape features are available with definitions in the CellProfiler manual (http://cellprofiler.org/manuals/) under MeasureObjectSizeShape. Nuclear features were similarly analyzed, and no differences were observed between different polarization states.
Processing time was approximately 2 h per plate tor 1536 images on a 12-core workstation running 12 CellProfiler workers in parallel.

After 48 hours, cells were fixed with formaldehyde (30 min, 37° C.) and stained for actin (5.0 μg mL⁻¹ DyLight 554 Phalloidin, Cell Signaling Technology), cell membrane (5.0 μg mL⁻¹ Alexa Fluor 647 wheat germ agglutinin, Thermo Fisher) and nuclei (DAPI, Invitrogen) for 25 min at room temperature. Plates were washed by PBS prior to imaging on a custom Olympus-based automated high-content screening microscope. Four images were acquired per well in a 2×2 grid, imported into CellProfiler (Broad Institute) for pre-processing and segmentation (Table 2).

TABLE 2

| Drug | Target(s) | Concentration range |
|---|---|---|
| GW2680 | CSF1R | 10 μM-100 pM |
| CEP32496 | CSF1R | 6 μM-60 pM |
| BLZ945 | CSF1R | 6 μM-60 pM |
| OSI930 | CSF1R/CKIT | 10 μM-100 pM |
| PLX3397 | CS1R/CKIT | 10 μM-100 pM |
| DASATINIB | Abl/Src/CKIT | 10 μM-100 pM |
| SUNITINIB | PDGFR/VEGFR/CKIT | 10 μM-100 pM |
| ABT860 | VEGF/PIXGF/KDR/CSF1R | 6 μM-60 pM |
| IMATINIB | Abl/CKIT/PDGFR | 10 μM-100 pM |
| FORETINIB | HGFR/VEGFR | 10 μM-100 pM |
| XL228 | IGFR/FHFR | 10 μM-100 pM |
| GEFTINIB | EGFR | 10 μM-100 pM |
| PD0325901 | MEK | 10 μM-100 pM |
| TRAMETINIB | MEK | 10 μM-100 pM |
| BENTAMAPIMOD | JNK | 10 μM-100 pM |
| DABRAFENIB | BRAF | 10 μM-100 pM |
| VEMURAFINIB | BRAF | 10 μM-100 pM |
| CRIZOTINIB | ALK/ROS1 | 10 μM-100 pM |
| UNC2025 | MERTK | 10 μM-100 pM |
| INDOXIMOD | IDO | 10 μM-100 pM |
| CELECOXIB | COX2 | 10 μM-100 pM |
| RAPAMYCIN | mTOR | 10 μM-100 pM |
| NIK12192 | V-ATPase | 10 μM-100 pM |
| TRICHOSTATIN A | HDAC | 10 μM-100 pM |
| IBET151 | BET | 10 μM-100 pM |
| TMP195 | HDAC | 10 μM-100 pM |
| BYL719 | PI3Ka | 10 μM-100 pM |
| GDC0941 | PI3Ka/g | 10 μM-100 pM |
| BKM120 | PI3K | 10 μM-100 pM |
| R846 | TLR7/8 | 10 μM-100 pM |
| MOTOLIMOD | TLR8 | 10 μM-100 pM |
| GS9620 | TLR7 | 10 μM-100 pM |

Computational cell classification was performed in CellProfiler Analyst (Broad Institute) by random forest assignment. Training data (examples provided, Supplementary Figure S1) consisted of approximately 50 healthy cells each representing undifferentiated (M0), M1-like, or M2-like phenotypes. The fast gentle algorithm was trained on the selected cells for unsupervised determination of weights and thresholds for cell shape features. The resulting set of parameters was used to score all other images. The enrichment score for M1 cells was output back into the database and imported into KNIME to generate per-well and per-treatment averages.

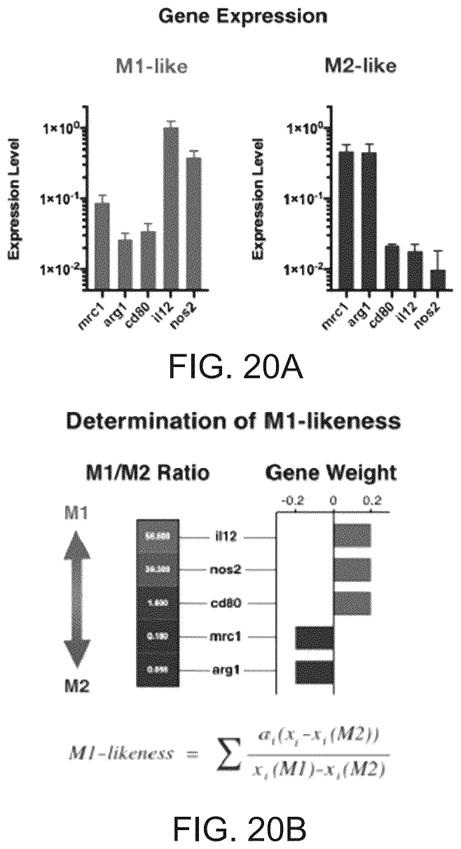

For transcriptional analysis, derived murine macrophages were treated with 10 ng mL⁻¹ recombinant mouse IL-4 (PeproTech 214-14) for 24 hours to induce an M2-like polarization state and subsequently dosed with fresh media supplemented by pharmacologic drugs at the prescribed concentrations. Macrophages treated only with IL-4 (10 ng mL⁻¹) or LPS (100 ng mL⁻¹) and IFNg (50 ng mL⁻¹) served as internal controls for M2-like and M1-like transcription profiles, respectively. After 24 hours, RNA was isolated by standard protocols (QIAGEN 74106) and subject to reverse transcription (Thermo Fisher 4368814) and qPCR (Thermo Fisher 44-445-57) for analysis of hrpt (Mm01545399_m1), arg1 (Mm00475988 m1), mrc1(Mm01329362_1), cd80 (Mm00711660_m1), il12b (Mm01288989_m1), and nos2 (Mm00440502_ m1). For analysis of human macrophages, cells were similarly treated and processed prior to analysis for expression of β-actin (Hs01060665_g1) and il12b (Hs01011518_m1). Data are presented as the gene expression (fold change relative to hprt or/β-actin, as indicated) or M1-likeness, calculated as described in Example 2, FIG. 20B.

### Nanoparticle Synthesis and Characterization

Polyglucose (succinyl-β-cyclodextrin (CD) or 10 kDa carboxymethyl dextran (5% carboxylated, TdB), 1.0 eq. carboxylate), N-(3-Dimethylaminopropyl)-N′-ethlycarbodiimide hydrochloride (Sigma; 10.0 eq. to carboxylate), and N-hydroxysuccinimide (Sigma, 5.0 eq. to carboxylate) were combined and dissolved in MES buffer (50 mM, pH 6.0) at the desired glucose concentration (1.25 to 20.0 % wt/v). The reaction was stirred for 30 min at room temperature prior to the addition of L-lysine (0.5 eq. to carboxylate, unless otherwise noted) and overnight crosslinking. The product was recovered by addition of brine (0.05 volumetric equivalents) and precipitation from a 10-fold excess of iced ethanol. Upon re-dissolution in water, the product was concentrated by centrifugal filtration (10 kDa MWCO, Amicon), washed repeatedly by water, passed through a 0.22. μM spin filter (Costar, Spin-x), and lyophilized. The final products were re-dissolved at a concentration of 20 mg mL⁻¹ prior to use. Particle size was calculated by dynamic light scattering (Malvern, Zetasizer APS) at a typical concentration of 5 mg mL ⁻¹ in 100 mM PBS. Zeta potential was determined at 100 μg mL⁻¹ in 10 mM PBS (Malvern, Zetasizer ZS) following calibration measurements on manufacturer standards. For scanning electron microscopy, samples were prepared at 1.0 μg mL⁻¹ in water, spotted on silica wafers, freeze-dried and sputter coated prior to imaging. Analysis of R848 affinity for CD was performed by a standard colorimetric competitive binding assay. Briefly, phenolphthalein (200 mM) was freshly prepared in 125 mM carbonate buffer (pH 10.5). Decrease in absorbance at 550 nm due to nanoparticle-phenolphthalein complexati on and absorbance recovery due to R848 competitive binding were measured (Tecan, Spark), and results are presented as absorbance relative to nanoparticle-free controls. The dissociation constant, K_{D}, was determined by treatment of β-cyclodextrin by increasing concentrations of R848 and fit to a one-site competitive inhibition model in GraphPad Prism 6 (GraphPad Software, Inc.). Drug loading in CDNP-R848 was analytically determined as a function of the molar ratio of R848 to CD in the nanoparticle, assuming the appropriate reaction equilibrium for one-to-one association: KD=[R848]*[CD]/[CD-R848], where [R848] is the concentration of unbound R848, [CD] is the concentration of unbound cyclodextrin in the nanoparticle, and [CD-R848] is the concentration of R848 bound by the nanoparticle. A molar ratio of guest-to-host ranging from 0.01 to 100 was examined, and drug loading (% wt/wt) was defined as 100*(M_{R848}/(M_{R848}+M_{CDNP})), where M_{CDNP} is the mass of nanoparticle, and M_{R848} is the mass of R848 bound by cyclodextrin.

### Fluorescence Derivatization

For intravital imaging and assessment of biodistribution, cyclodextrin nanoparticles were fluorescently labeled. The CDNP nanoparticle was dissolved at 20 mg mL⁻¹ in carbonate buffer (0.1 M, pH 8.5) prior to addition of VivoTag 680 XL (PerkinElmer, 1.0 mg mL⁻¹ in anhydrous DMSO) at a final concentration of 50 μM. The reaction was allowed to proceed for 3 hours at room temperature prior to product recovery by centrifugal filtration (10 kDa MWCO, Amicon), repeated washing by water to remove unbound dye, and lyophilization. Resultant CDNP-VT680 was re-dissolved at a concentration of 10 mg mL⁻¹. Absorption at 668 nm (Nanodrop) was used to determine the label concentration (1.79±0.03 nM mg⁻¹) by the Beer-Lambert equation, (A=εbc, where A is the absorbance, ε is the molar absorptivity 210,000 M⁻¹cm⁻¹, and c is the concentration).

### Pharmacokinetic and Biodistribution Analysis

The blood half-lite of CDNP-VT680 was determined by time-lapse confocal fluorescence microscopy of vessels in the ear during and immediately following tail vein injection of Pacific Blue Dextran and CDNP-VT680 (0.5 mg). Time-lapse images were acquired continually over the first 3 hours after CDNP-VT680 injection, after which the mice were allowed to recover before subsequent imaging at 24 hours. Across three separate C57BL/6 mice, multiple fields of view were analyzed by identification of regions of interest within the labeled vasculature. Mean fluorescence intensity was determined as a function of time, background subtracted, and normalized to the to peak fluorescence intensity. Resulting data was fit to a mono-exponential decay in GraphPad Prism 6,

At 24 hours following injection, examination of CDNP biodistribution was performed. Surgically resected tissues of interest were thoroughly washed in PBS, weighed, and placed in an OV110 (Olympus) for brightfield imaging to identify regions of interest and fluorescence reflectance imaging (1000 ms exposure time; λₑₓ=620-650 nm, λₑₘ₌680-710 nm). Integrated fluorescence density was determined for ROIs representing each tissue (ImageJ, NIH). Values were background-subtracted for tissue autofluorescence by imaging of corresponding tissues from a vehicle treated control. Percentage of injected dose was determined relative to standards of CDNP-VT680 prepared in 1.0% intralipid (McKesson, 988248), to account for optical scattering of tissue, and values are presented following normalization to tissue mass.

### Intravital Microscopy

Intravital examination CDNP-VT680 distribution into macrophages and tumor cells was examined using dorsal skinfold window chambers installed on recently developed MerTK-GFP mice inoculated with MC38-H2B-mApple. Mice received CDNP-VT680 i.v. (0.5 mg) 24 hours prior to imaging. Intravital examination of IL12 expression was similarly performed using p40-IRES-eYEP IL12 reporter mice (#015864, Jackson). Prior to imaging, mice received intravenous administration of R848 (2.0 mg kg⁻¹), CDNP-VT680 (16.5 mg kg⁻¹ CDNP), or CDNP-VT680+R848 (16.5 mg kg⁻¹ CDNP-VT680+2.0 mg kg⁻¹ R848; 1/1.1 R848/CD molar ratio). IL12 expression was examined at 24 hours following treatment. In both cases, macrophages and vasculature were labeled by Pacific Blue-ferumoxytol and Pacific Blue-dextran, respectively. Images were acquired on a FV1000MPE confocal imaging system (Olympus). Pacific Blue, GFP/YFP, mApple, and VT680 were imaged sequentially using 405-, 473-, 559-, and 633-nm light sources and BA430-455, BA490-540, BA575-620, and BA575-675 emission filters with DM473, SDM560, and SDM640 beam splitters.

Images were pseudo-colored and processed in FIJI (ImageJ, NIH) by adjusting brightness/contrast, creating z-projections of image stacks, and performing a rolling ball background subtraction. For quantification of IL12 expression, the sum of YFP, Pacific Blue, and VT680 channels were segmented by automated thresholding using the RenyEntropy method to generate a mask and corresponding ROIs for individual macrophages. The fluorescence intensity was determined for YFP within each ROI, and data are presented following normalization to the average intensity for CDNP control treatment.

Flow Cytometry For examination of CDNP-VT680 biodistribution in MerTK-GFP mice, MC38 tumors and tissues of interest were excised 10 days after tumor implantation, 24 hours after intravenous injection of CDNP-VT680 (0.5 mg). For examination of IL12 expression, MC38 tumors were harvested 9 days after intradermal implantation into IL12-eYFP mice, 24 hours following intravenous administration of R848 (2.0 mg kg⁻¹), CDNP-VT680 (16.5 mg kg⁻¹ CDNP), or CDNP-VT680±R848 (16.5 mg kg⁻¹ CDNP-VT680±2.0 mg kg⁻¹ R848; 1/1.1 R848/CD molar ratio) in saline. Tissues were minced, incubated in RPMI containing 0.2 mg mL⁻¹ collagenase I (Worthington Biochemical) for 30 min at 37° C. and then passed through a 40 μm filter. Red blood cells were lysed using ACK lysis buffer (Thermo Fisher Scientific) prior to pre-treatment with low affinity Fe receptor blocking reagent (Tru Stain FcX anti-CD16/32 clone 93, BioLegend) and staining in phosphate buffered saline containing 0.5% BSA and 2 mM EDTA with fluorochrome labeled antibodies against CD45 (30-F11, eBioscience), CD11c (N418, BioLegend), Ly6G (1A8, Biolegend), F4/80 (BM8, BioLegend), and 7-AAD. Samples were run on a LSR II flow cytometer (BD) and analyzed in FlowJo v.8.8.7 (Tree Star, Inc.) to identify macrophages (CD45+MerTK+Ly6G-F4/80+) in IL12-eYFP mice as well as macrophages (CD45+MerTK+Ly6G−), neutrophils (CD45+MerTK-Ly6G+), and other immune cells (CD45+) in MerTK-GFP mice. Identically treated tissue from MC38 tumors grown in wild type C57BL/6 mice served as a control for thresholding cutoffs for IL12+ and CDNP-VT680+ cells in analysis of IL12-eYFP induction.

### Tumor Growth Models

Tumor growth studies were initiated by intradermal injection of 2×10⁶ MC38 cells suspended in 50 μL of PBS. Tumors were allowed to grow to 25 mm² (8 days) at which time treatment cohorts were assigned such that tumor size and body weight were normalized across groups at baseline. For repeated dosing experiments, animals were treated 3 times weekly by i.v. administration of R848 (2.0 mg kg⁻¹), CDNP (16.5 mg kg⁻¹), or CDNP (16.5 mg kg⁻¹) with R848 (2.0 mg kg⁻¹) in saline. For single dosing experiments, animals were treated by i.v. administration of R848 (3.0 mg kg⁻¹), CDNP (24.6 mg kg⁻¹), or equivalent dosing of CDNP (24.6 mg kg⁻¹) with R848 (3.0 mg kg⁻¹) in saline. For aPD-1 treatment, the 29F.1.A12 aPD-1 clone was administered at a dose of 200 μg by intraperitoneal injection. At set time points, tumor growth was assessed by caliper measurement (A=length×width) and values are reported following normalization to area at the time treatment was initiated.

### Statistical Analysis

Data are presented as mean±standard error unless otherwise indicated. Statistical analyses were performed using GraphPad Prism 6 (GraphPad Software Inc.). Statistical significance was determined by analysis of variance, using repeated measures where appropriate, in conjunction with post hoc Tukeys HSD. For in vivo studies of tumor growth, temporal comparisons were made by Friedmans test and comparison at set time points were performed by Kruskal-Wallis, each using post hoc Dunn's test for multiple comparisons. Survival analysis was performed by log-rank test. Significance was determined at P=0.05.

### Example 1. Strategy for High-Content Screening the Therapeutic Re-Education Macrophages.

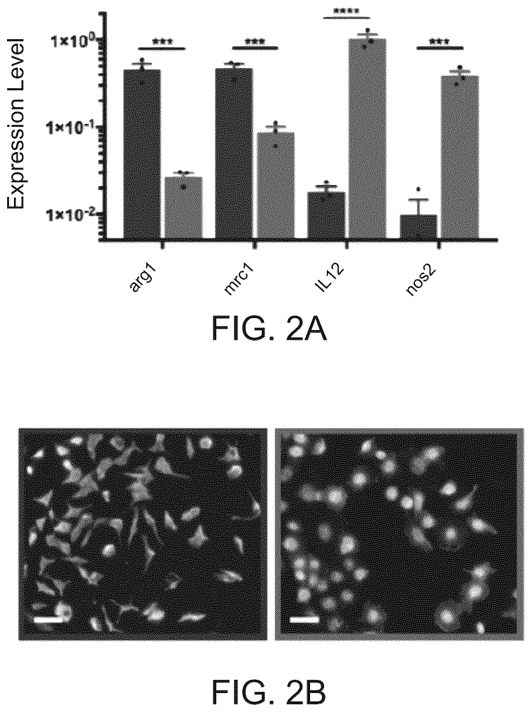

FIG. 2A depicts gene expression of M2-like (IL-4 treated; left bars) and M1-like (LPS/INFγ treated; right bars) polarization states. Data represent mean±s.e.m. of fold change relative to hprt, N=3. ***P<0.0005, ****P<0.00001 (two-way ANOVA, Fisher LSD). Tumor-associated macrophages (TAMs) often represent a dominant proportion of the immune cell infiltrate and predominantly assume a tumor supportive M2-like signature that includes the expression of mannose receptor-1 (MRC1) and the metabolic checkpoint enzyme arginase-1 (ARG1; FIG. 2A). In contrast, classically activated (M1-like) cells are often characterized by the expression of nitric oxide synthase (NOS2) and interleukin 12 (IL12) (FIG. 2A). FIG. 2B depicts gross observation of cell shape for M2- and M1-like polarization states. Scale bar: 50 μm. It was observed that macrophage polarization states demonstrate hallmark morphology, including elongated projections for M2-like cells (left image) as opposed to a round and flattened morphology for their M1-like counterparts (right image). Despite these characterizations, macrophage populations exhibit significant heterogeneity, limiting population-based analysis.

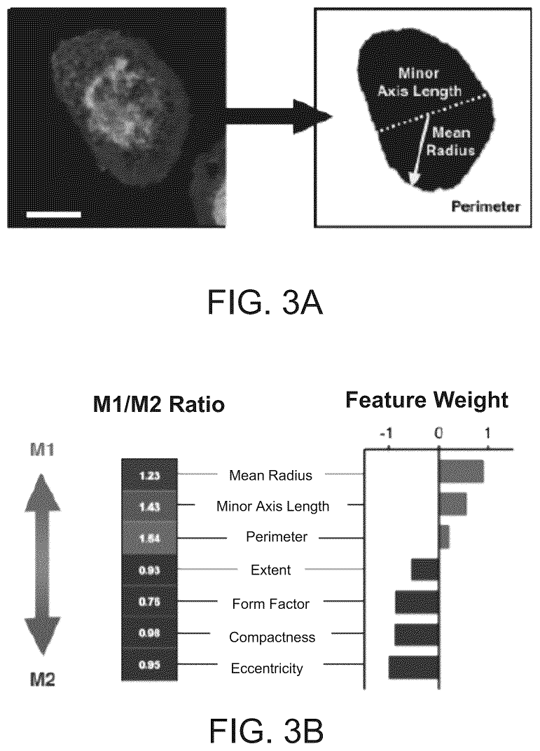

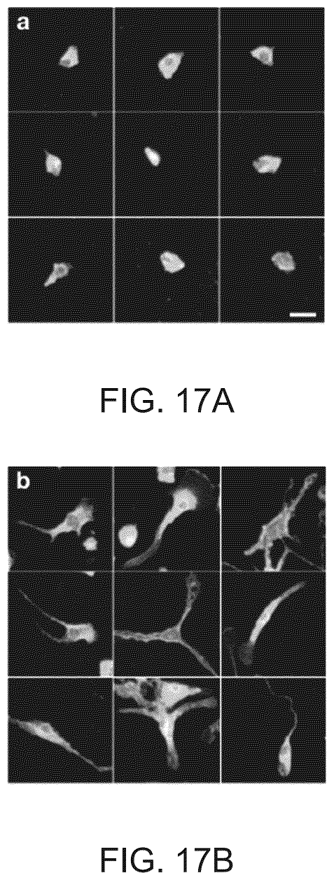

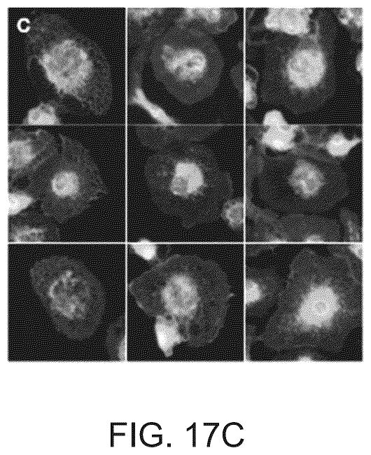

Cell morphology was used as an integrated biomarker of cell function, including as an indicator for age and immunosuppressive capacity. The method that was used uses high-content image analysis via computational automated segmentation to extract features of single cells such as cellular radius, axis lengths, compactness, and eccentricity which are associated with the polarization state. FIG. 3A depicts raw images that were processed by automated segmentation, allowing measurement of prominent features in identification of M1-like polarization include mean radius (solid line), minor axis length (dotted line), and perimeter (dashed line). Scale bar: 25 μm. Subsequent analysis utilized unbiased classification of polarization phenotypes, where computationally assigned shape-feature weights were determined from supervised training data sets (FIGS. 17A-C which depict undifferentiated (M0) cells, M-CSF-treated. M2-like cells, and LPS/IFN-treated M1-like cells, respectively). Morphological phenotyping was conducted by random forest assignment (FIG. 3B), where feature weights determined are reflective of the relative differences in M1-like and M2-like training sets, reflecting the relative changes in cell-shape features between M1- and M2-like polarization states (FIG. 3B). The proportional increase in M1-like cells within the examined population is expressed as M1 enrichment. Table I depicts the CellProfiller pipeline used for quantification of cell shape features.

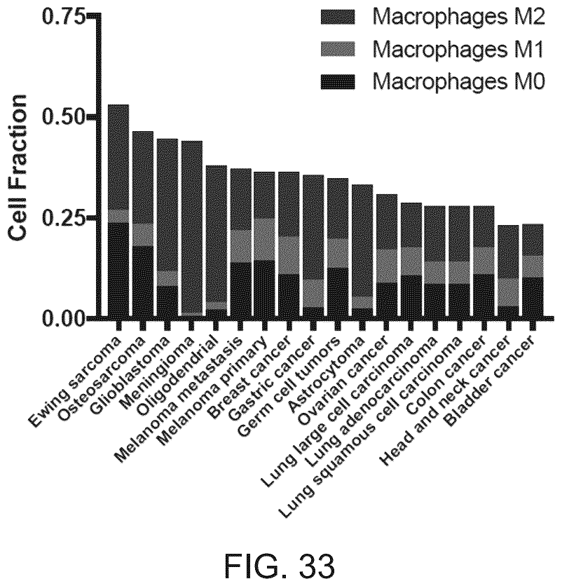

FIG. 33 depicts the percentage of M2, M1, and M0 cells present in various cancer types. Most cancer types, particularly meningioma and oligodendrial cancer, exhibit a high percentage of M2 macrophages relative to other cancer types.

### Example 2. In Vitro Assessment of Macrophage Phenotype.

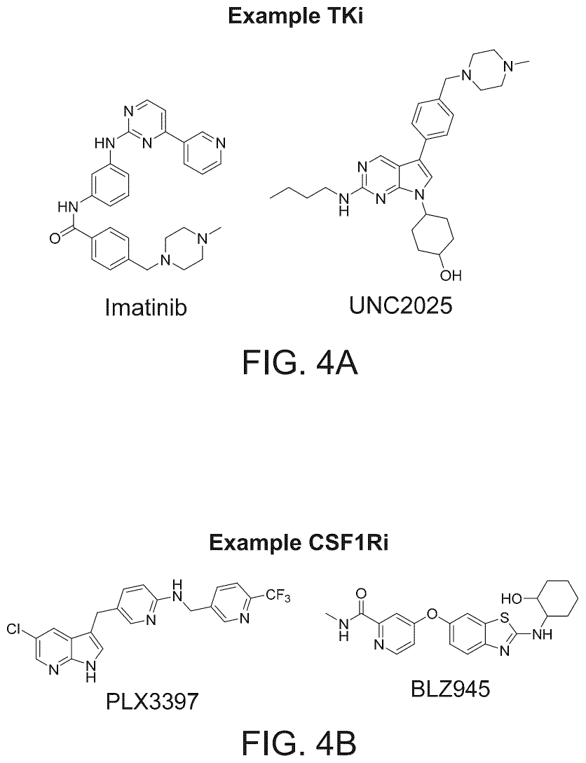

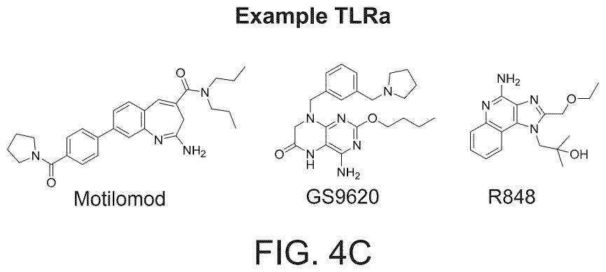

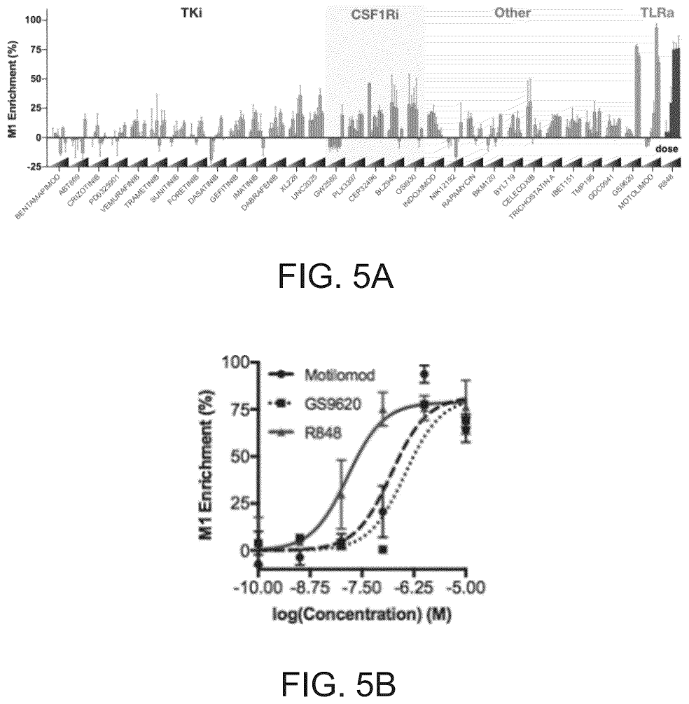

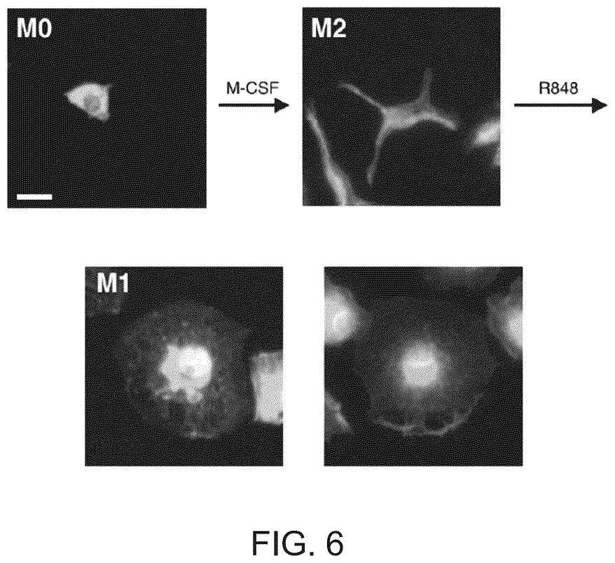

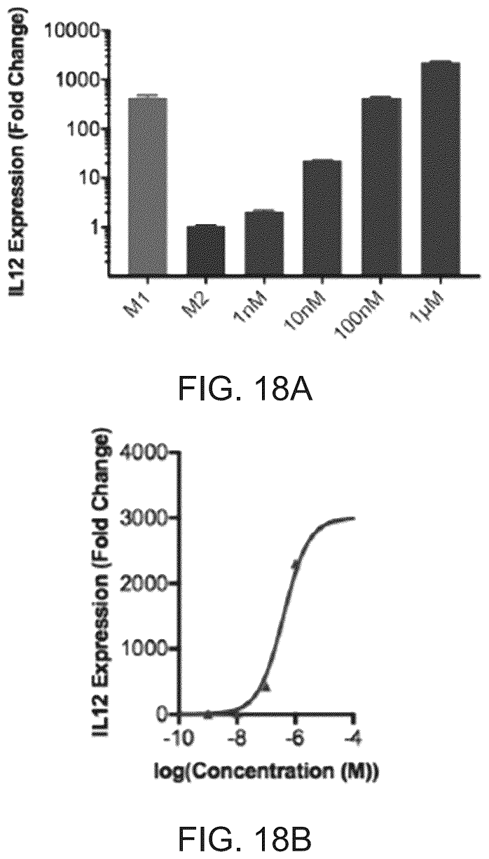

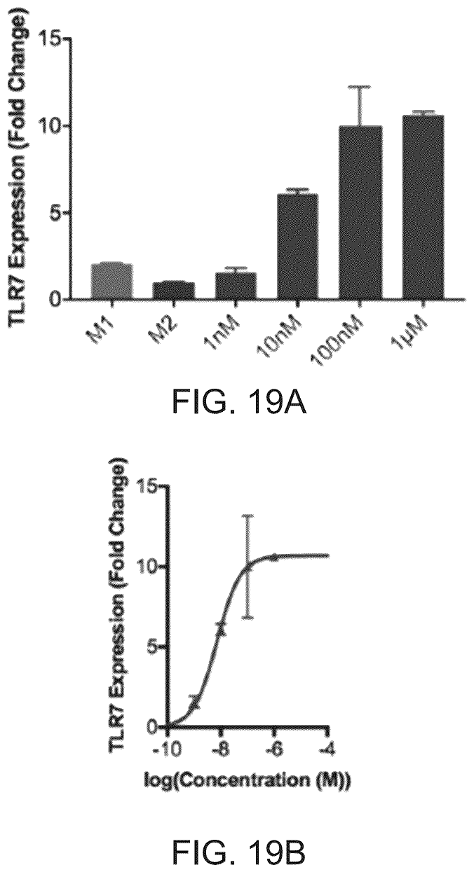

Having established a HTS for examination of cell state, drugs capable of macrophage re-educating were then identified. A panel of 38 drugs was curated from the literature, representing specific drugs or drug classes which have been implicated in macrophage polarization. FIGS. 4A-C depicts general classification and examples of drugs of interest, including tyrosine kinase inhibitors (TKi; FIG. 4A), colony-stimulating factor 1 receptor inhibitors (CSF1Ri; FIG. 4B), and toll-like receptor agonists (TLRa; FIG. 4C). Table 2 lists additional drugs and/or drug classes that were used. Freshly isolated murine monocytes were differentiated into an M2-like phenotype, followed by drug treatment spanning six orders of magnitude in drug concentration. FIG. 5A depicts a morphological determination of M1 enrichment in response to drug treatment at variable concentrations, allowing stratification of drug activity. Macrophage colony-stimulating factor 1 receptor (CSF1R) activation is a driving signal in M2-like polarization, and experimental CSF1R inhibition is known to bias macrophage polarization in addition to altering TAM recruitment and distribution in vivo. With the exception of GW2580, inhibitors of CSF1R demonstrated enrichment of the M1 population in a dose dependent manner. Similar enrichment was observed in the M1 population with a number of tyrosine kinase inhibitors, often to a lesser degree, including for imatinib (Bcr-Abl), dabrafenib (B-raf), gefitinib (EGFR), XL228 (IGF1R), and UNC2025 (MerTK). However, the largest polatization effects were observed for agonists of the pattern recognition receptors toll-like receptor 7 and 8 (TLR7/8). Agonists including motolimod (TLR7 specific), GS9620 (TLR8 specific), and the TLR7/8 agonist R848 (i.e., resiquimod) yielded M1 enrichments which were as pronounced as standard M1 induction by LPS and IFNγ. FIG. 5B depicts dose response of M1 enrichment in response to TLR agonists. Data represent mean±s.e.m., N=2 independent experiments, n>100 cells per condition. In a direct comparison of the latter three TLR agonists, R848 emerged as the most potent driver of macrophage re-education, with an EC₅₀ of 14.1 nM, an order of magnitude improvement relative to TLR7 and TLR8 specific agonists examined. FIG. 6 depicts representative images of in vitro M2-like macrophage derivation and subsequent re-education by R848 (48 hours, 100 nM). Right: M1-like (LPS/IFNγ treated) cell, provided for comparison. Scale bar: 25 μm, In vitro, murine macrophages re-educated by R848 closely resembled M1 controls. M2-like (IL-4 treated) macrophages by were cultured in the presence of R848 (24 hours). FIGS. 18A-B show IL12 transcription indicates response to nM concentrations of R848. FIGS. 19A-B show TLR7 expression increases in response to agonist, likely sensitizing response to drug treatment and may mechanistically explain its potency. IC50 values (IL12: logIC50=−6.40±0.051; TLR7: logIC50=−8.14±0.17) are in good agreement with those obtained for M1 enrichment by high-content screening approaches (logIC50=−7.84±0.159). Expression values represent fold change relative to M2 controls, N=3.

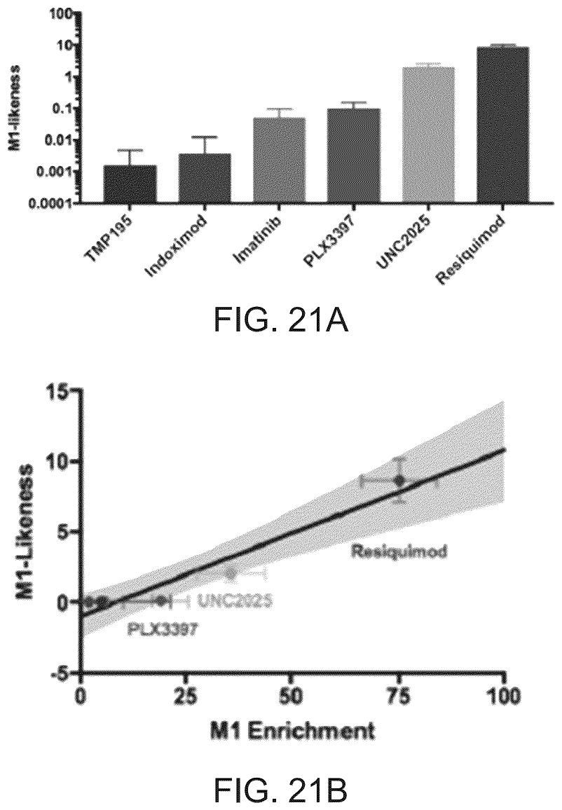

A subset of drugs having a range of M1 enrichment activities were further scrutinized by qPCR analysis of representative M1-like (nos2, il12, and cd80) and M2-like (mrc1, arg1) transcripts. FIG. 20A is a set of two bar graphs depicting gene expression of M1-like and M2-like macrophages, expressed as fold change relative to hprt. N=3. FIG. 20B shows simultaneous examination of multiple statistically and biologically relevant transcriptional markers provides a metric of phenotype: M1-likeness. Determination of M1-likeness, accounting for both canonical M1- and M2-like gene expression levels (xᵢ) with assigned gene weights (aᵢ). M1-likeness is determined by summing the changes in expression for each gene resulting from treatment, normalized to the range of expression observed in the training data. FIG. 21A is a bar graph that depicts transcriptional M1-likeness in response to a select validation set of drugs, indicating the strong ability of R848 to induce M1-like gene transcription. FIG. 21B shows the correlation of morphological and transcriptional phenotyping scores, indicative of the ability of high-content screening (M1 enrichment) to accurately predict macrophage phenotype (M1-likeness). Black line: linear fit with 95% CI (shaded); R²>0.92. These studies validated the ability of M1 enrichment to predict expression of an inflammatory transcriptome, indicative of M1 activation.

### Example 3. Development and Characterization of Cyclodextrin Nanoparticles (CDNPs).

It is understood that certain dextran nanoparticles have native macrophage avidity which results in rapid, preferential distribution to TAMs relative to other cells present in the TME. β-cyclodextrin (CD) shares similar chemical composition with linear dextran, suggesting potential for macrophage avidity. Moreover, host-guest inclusion by macrocycles, such as CD, is an established mechanism for drug solubilization and nanoparticle-mediated drug delivery which forgoes chemical modification of established drug compounds (see Zhang & Ma, Nature Protocols, 2016, 11:1757); and Rodell et al, Bioconjug Chem. 2015, 26:2279-2289). The interaction of CD with R848 was therefore used to enable formation of drug-loaded nanoparticles.

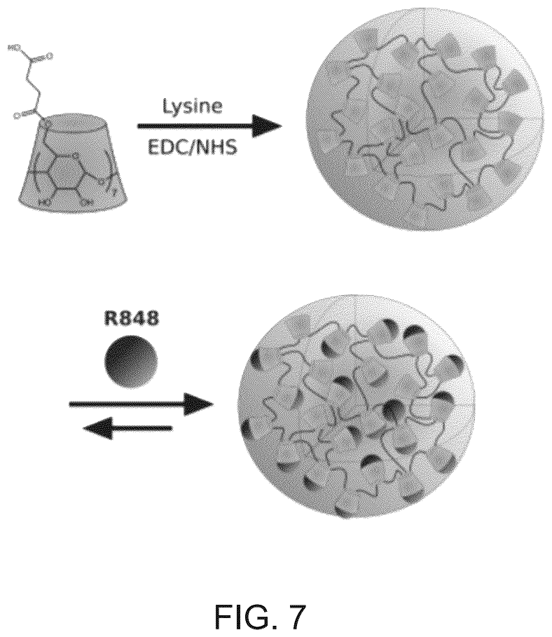

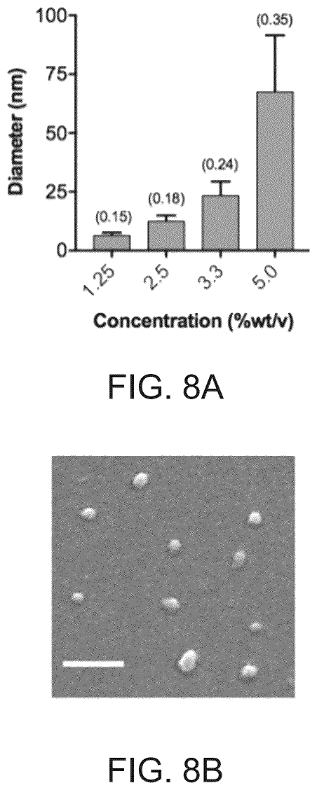

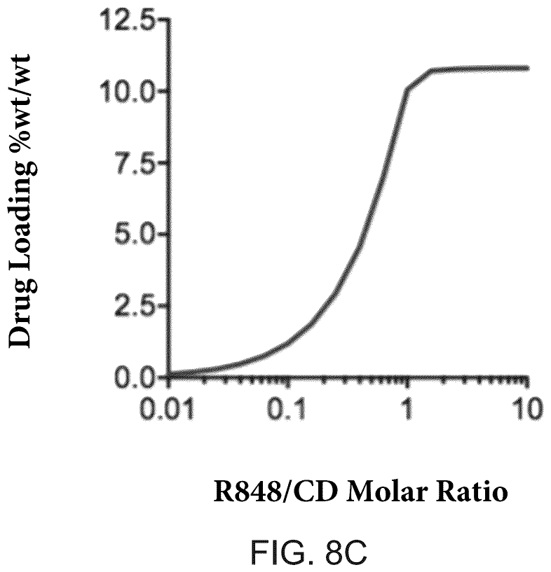

FIG. 7 depicts a schematic of cyclodextrin nanoparticle (CDNP) preparation by lysine crosslinking of succinyl-β-cyclodextrin and subsequent drug loading by guest-host complexation of R848. FIG. 8A depicts dynamic light scattering measurement of hydrodynamic diameter, dependent on the concentration of CD in solution during crosslinking. Polydispersity index (PDI) is indicated in parentheses. FIG. 8B depicts a scanning electron microscopy image of CDNPs. Average diameter: 29.3±1.70 nm. Scale bar: 200 nm. These results showed that nanoparticles with a diameter of approximately 30 nm, preferable for phagocytic uptake, were reliably synthesized overnight starting with a 3.3% _{wt/vol} solution of CD. A molar ratio of 1:2 L-lysine per succinyl group resulted in a zeta potential of −9.87±0.59 mV, as compared to 1:1 molar ratio which yielded near-neutral (0.90±1.90 mV) charge which is known to negatively impact macrophage phagocytosis and enhance undesirable hepatic uptake. Thus, the CDNP formed at 3.3% wt/v with a 1:2 lysine to succinyl ratio were employed in subsequent studies. FIG. 8C depicts a plot of drug loading (% wt/wt R848 relative to CDNP-R848) as a function of the molar ratio of guest-to-host. Results represent the mean loading calculated at reaction equilibrium. These results indicated a strong drug-nanoparticle interaction which potentiated high drug loading within the nanoparticle (10.39±0.20% wt/wt at a 1.1:1 ratio of CD to R848).

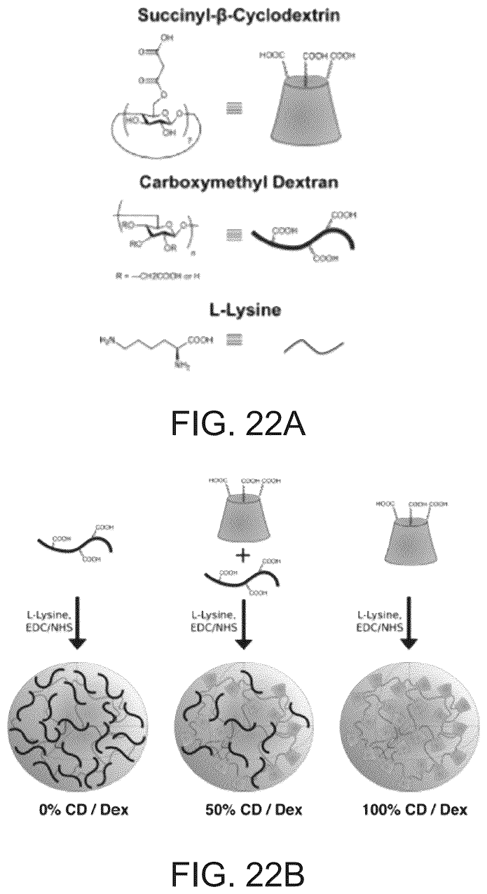

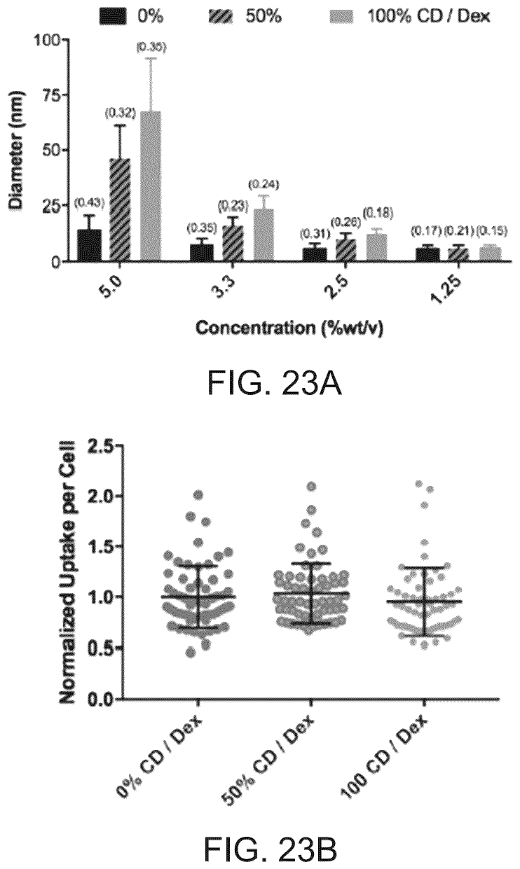

FIGS. 22A-22B depicts the formation of nanoparticles including succinyl-β-cyclodextrin (CD, orange) and carboxymethyl dextran (Dex, black) at defined ratios of 0%, 50%, and 100% CD/Dex by L-Lysine crosslinking. Under equivalent crosslinking conditions, CD contributed to formation of larger nanoparticles and reduction in polydispersity (FIG. 22B). FIG. 23A depicts a bar graph, showing that uptake of nanoparticles by RAW 264.7 cells in vitro was independent of particle composition (P>0.36, ANOVA), and indicating the use of CD as a base material does not inhibit phagocytosis relative to established dextran nanoparticles. FIG. 23B shows data normalized to the 0% CD/Dex condition. N>60 cells per condition. The data in FIGS. 22A-22B and 23A-23B show that the use of CD as a base material did not negatively impact nanoparticle phagocytosis relative to dextran-formulated controls.

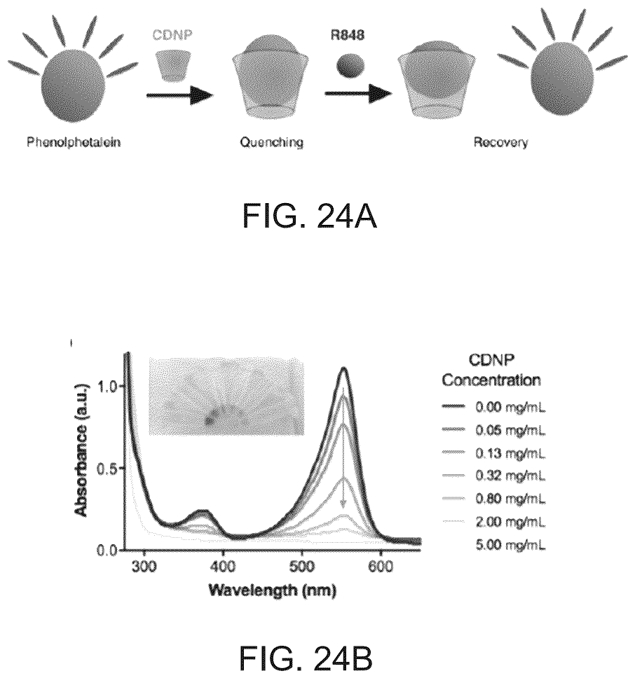

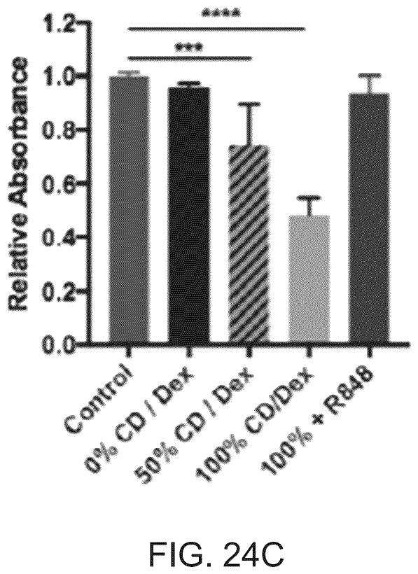

FIG. 24A is a schematic that shows the assessment of the ability of CDNPs to bind small molecules by a chromatographic competitive binding assay. FIG. 24B shows that phenolphthalein absorbance (550 nm) was quenched (indicated, arrow) in the presence of increasing CDNP concentrations. Data represent the average of three independent measurements. Inset: micrograph with increasing CDNP concentration from left to right. The plot lines correspond to a decrease in mg/mL, concentration with decreasing absorbance. FIG. 24C shows that quenching of absorbance (550 nm) in the presence of nanoparticle (0.5 mg mL⁻¹) was dependent on CD content (FIG. 24C). Addition of R848 (20 mM) competitively bound to the 100% CD/Dex particle, recovering phenolphetalein absorbance. Data are expressed as mean±s.d.; N=5; ***P<0.001, ****P<0.0001 (ANOVA, Turkey HSD) relative to nanoparticle free control, These data indicate that CD enabled drug-nanoparticle complexation.

### Example 4. In Vivo Biodistribution and Pharmacokinetics of CDNP.

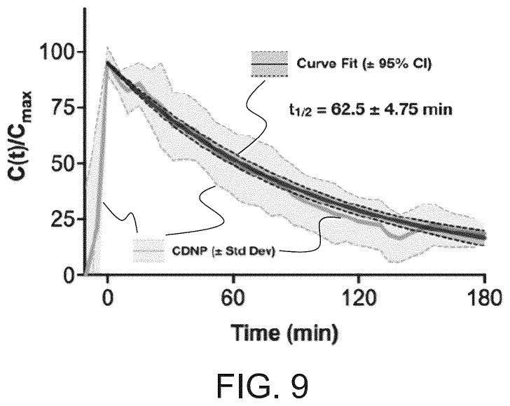

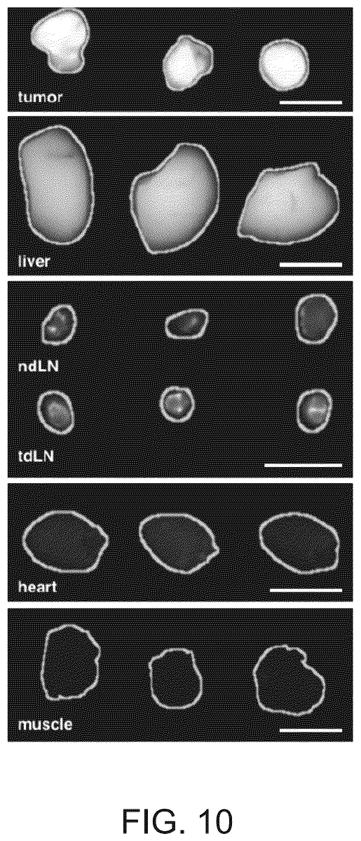

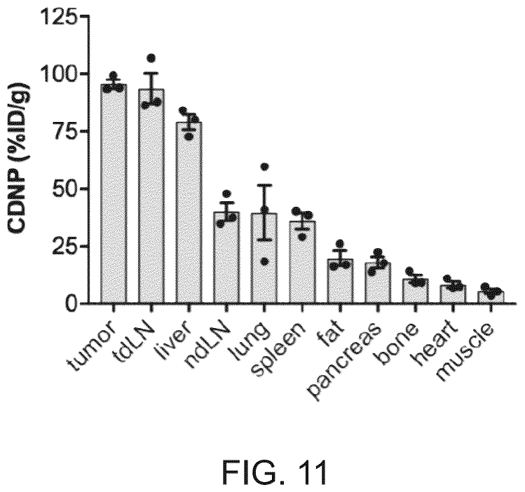

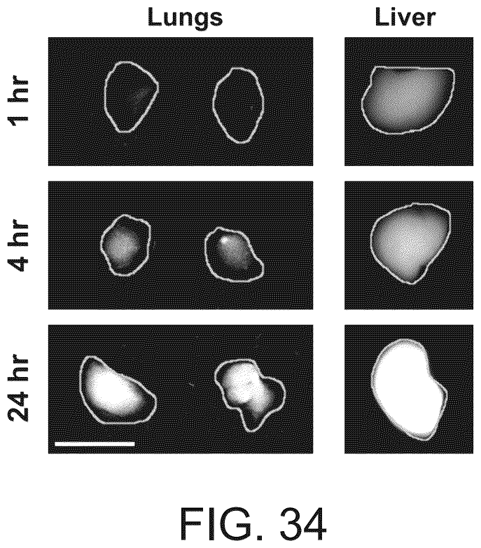

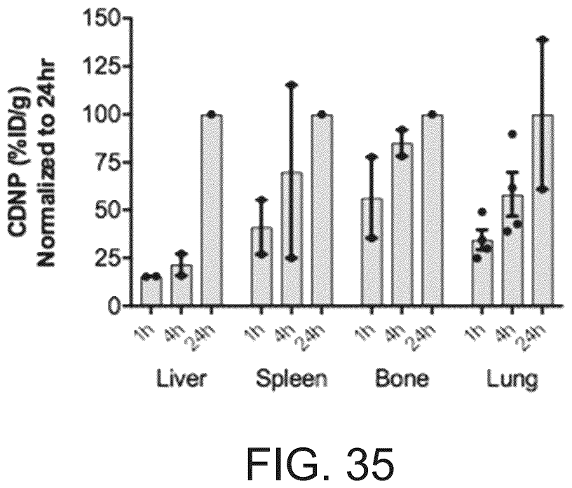

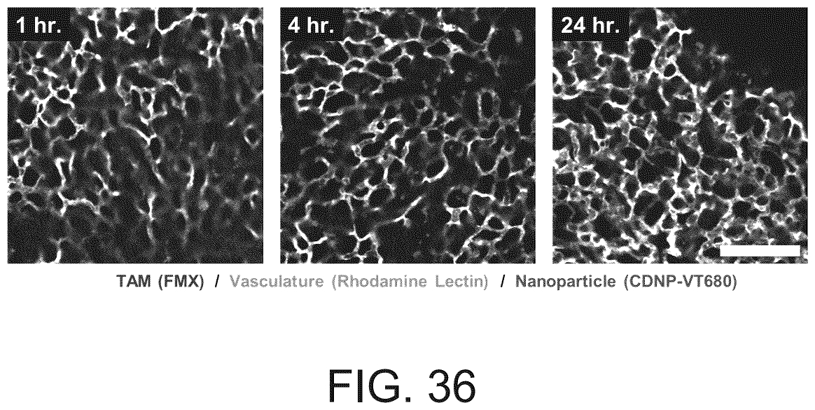

To examine the pharmacokinetics and biodistribution of the newly developed CDNP, a fluorescent derivative (CDNP-VT680) was developed, where the covalently bound fluorochrome readily allows for examination in vivo by fluorescence microscopy. Systemic circulation and biodistribution were examined in an immunocompetent mouse model of colorectal cancer (MC38) in C57BL/6 mice. First, time-lapse confocal fluorescence microscopy was performed of vessels within the ear for assessment of systemic circulation, demonstrating a vascular half-life (t1/2) of 62.5±4.75 min. FIG. 9 depicts a plot of nanoparticle blood half-life in MC38 tumor-bearing C57BL/6 mice, quantified by time-lapse confocal fluorescence microscopy of CDNP-VT680. Data represent mean±s.d. (shaded), N=3. Subsequently, organ biodistribution was examined by fluorescence reflectance imaging at 24 hours post-injection. FIG. 10 depicts fluorescence reflectance imaging of CDNP-VT680 accumulation in the tumor and representative organs at 24 hours following administration (λₑₓ₌620-650 nm, λₑₘ₌680-710 nm). Tissues are outlined (cyan) for clarity. Scale bars: 5.0 mm. FIG. 11 depicts corresponding quantified biodistribution of CDNP-VT680. Data are presented as mean±s.e.m., N=3. CDNP accumulation was highest in tumors (95.5±3.3% ID/g tissue) followed by draining lymph node (93.6±11.5% ID/g tissue). Retention in other RES organs was lower than within the tumor, including in liver (78.9±5.8% ID/g tissue) the spleen (35.9±6.0% ID/g tissue; FIG. 11). Similar examination was performed at earlier timepoints. FIG. 34 is a fluorescence reflectance image of CDNP-VT680 accumulation in the lungs and liver at 1, 4, and 24 hours following administration of the CDNP-VT680. FIG. 35 is a bar graph of CDNP-VT680 distribution in various tissue and organs at 1, 2, and 24 hours following administration of the CDNP-VT680. FIG. 36 is confocal fluorescence microscopy images of CDNP-VT680 accumulation in the lung at 1, 4, and 24 hours following administration. CDNP-VT680 co-localizes with macrophage signal within the lung, does not result in vascular casts, and increases in macrophages and macrophage rich tissues over time.

### Example 5. Uptake of CDNPs by Tumor Associated Macrophages.

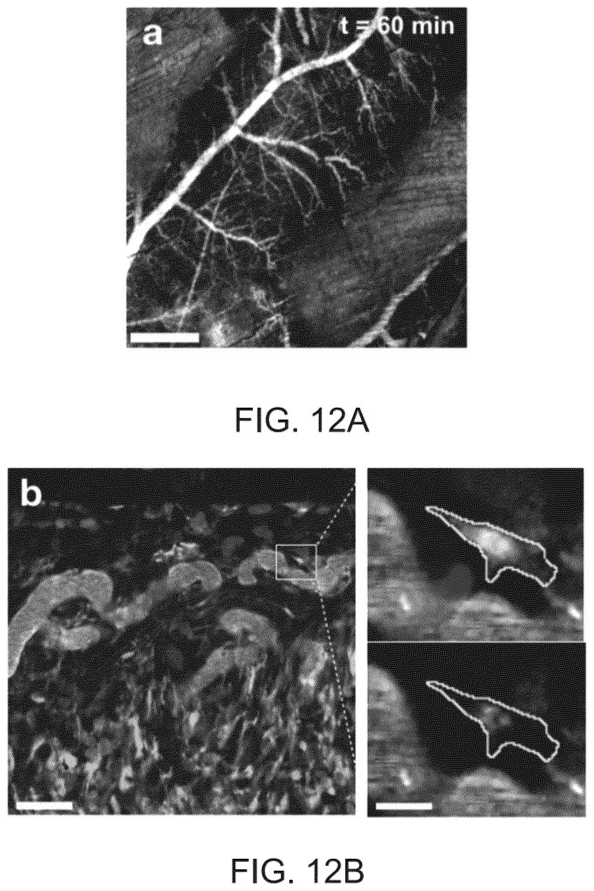

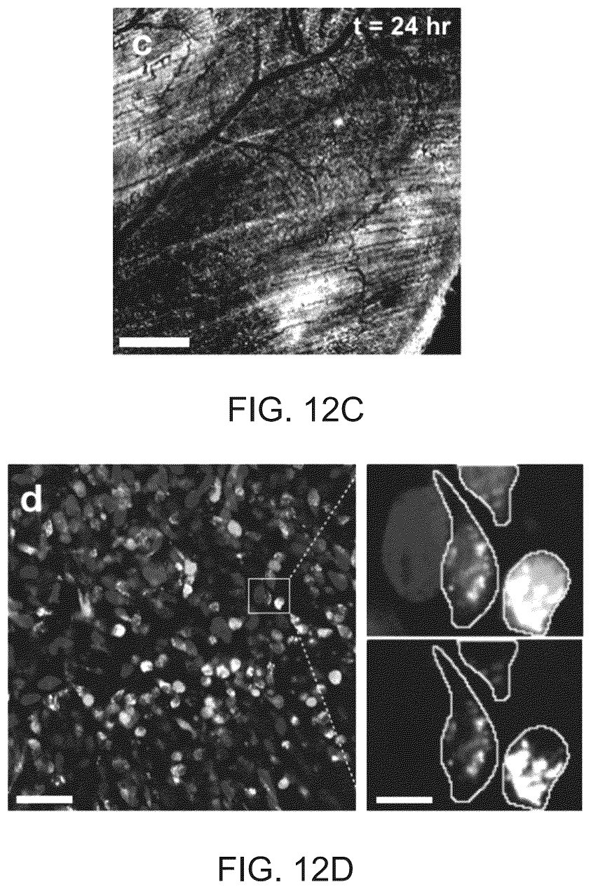

To further interrogate the intratumoral kinetics and cellular distribution, a dorsal window chamber setup was employed for intravital imaging. Tumors were generated by inoculation with 1×10⁶ MC38-H2B-mApple cells, allowing identification of tumor cells. To enable identification of TAMs, a CRISPR-CAS reporter mouse was used, wherein TAMs are detectable through MerTK-GFP expression. The distribution of CDNP-VT680 was examined by confocal fluorescence microscopy in a MerTK^{GFP/+} mouse bearing an MC38-H2B-mApple tumor in a dorsal window chamber model 60 min following administration (FIGS. 12A-12B. High magnification images; FIG. 12B is an expanded image) demonstrate rapid CDNP accumulation in perivascular macrophages within 60 minutes, outlined for clarity. FIGS. 12C-12D depict confocal fluorescence microscopy images 24 hours post-injection, showing that CDNPs were cleared from the vasculature and had accumulated within TAMS throughout the tumor. FIG. 12C shows that vascular clearance was observed, and FIG. 12D shows that CDNP is distributed to TAMs throughout the tumor site. Scale bars: 1.0 mm (a, c), 50 μm (b, d), and 10 μum.

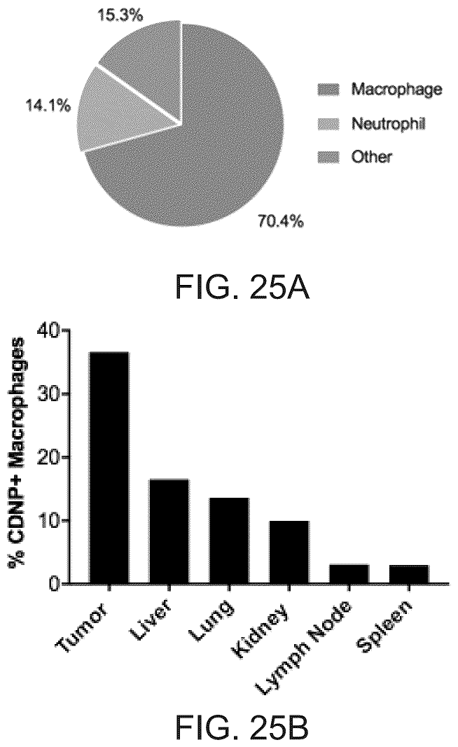

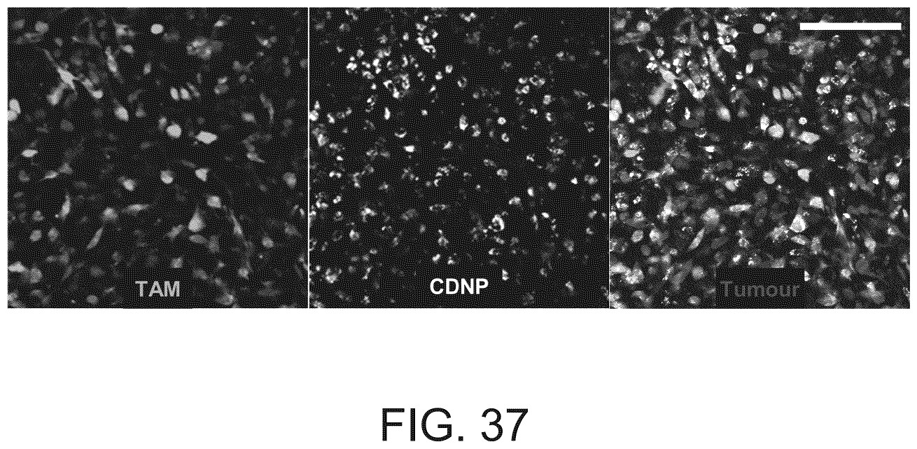

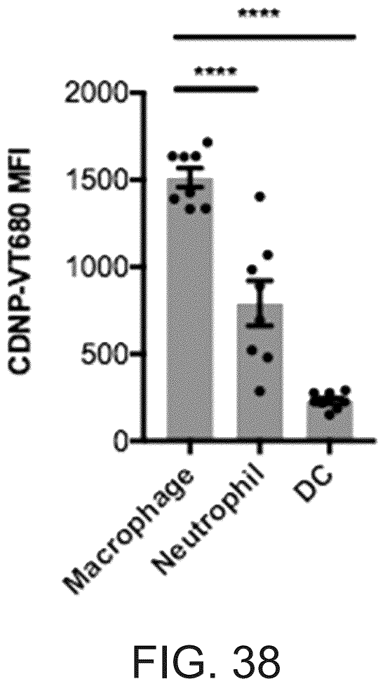

At 24 hours following administration of CDNP-VT680, relevant tissues were harvested from a MerTKGFP/+mouse bearing an MC38-H2B-mApple tumor, and flow cytometry was performed to identify the distribution (FIG. 25A) of CDNP-VT680 to immune cells, including macrophages (CD45+MerTK+Ly6G−), neutrophils (CD45+MerTK−Ly6G+), and other immune cells (CD45+). FIG. 25B shows the percentage of macrophages in each tissue examined that demonstrated high levels of CDNP-VT680 uptake. Confocal fluorescence imaging of similarly treated MC38-H2B-mApple tumors was performed. FIG. 37 is confocal fluorescence microscopy images of CDNP-VT680 24 hours following administration. FIGS. 38 is a bar graph of distribution of CDNP-VT680 in various immune cells in the tumor, These data show that CDNP accumulation was not observed in tumor cells, but rather mostly accumulated in macrophages.

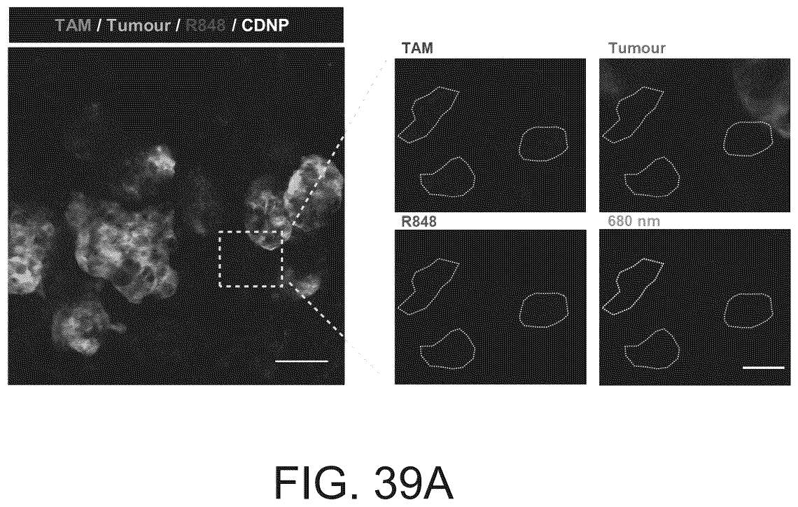

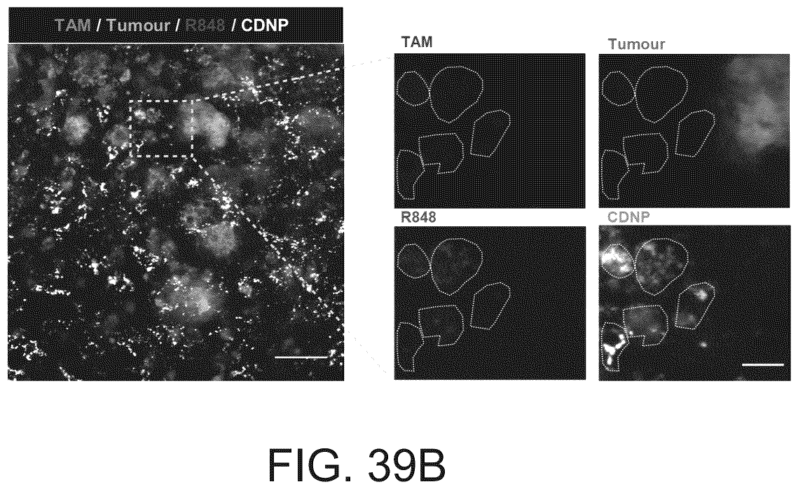

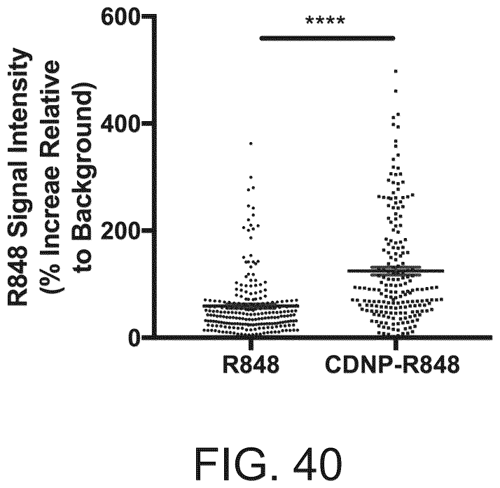

R848 delivery to TAMs was also examined in an orthotopic lung adenocarcinoma model (eGFP-expressing KrasG12D p53−/− mutant (KP) lung adenocarcinoma) by imaging of CDNP-VT680 and a newly developed fluorescent drug conjugate, R848-BODIPY TMR. FIGS. 39A, 39B, and 40 shows R848 and the CDNP carrier co-localized at the subcellular level within TAMs in vivo, and CDNP results in a near threefold increase in local drug concentration relative to solubilized R848 alone.

### Example 6. Intravital Re-Education of Tumor Associated Macrophages.

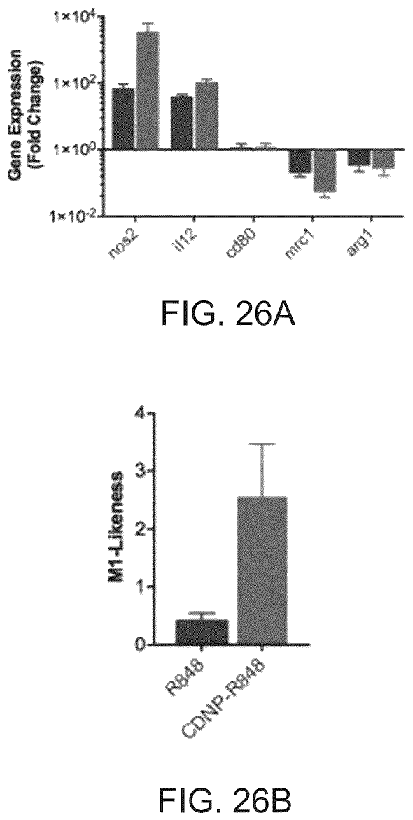

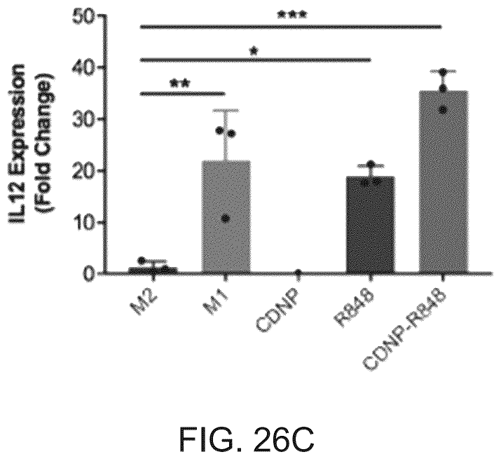

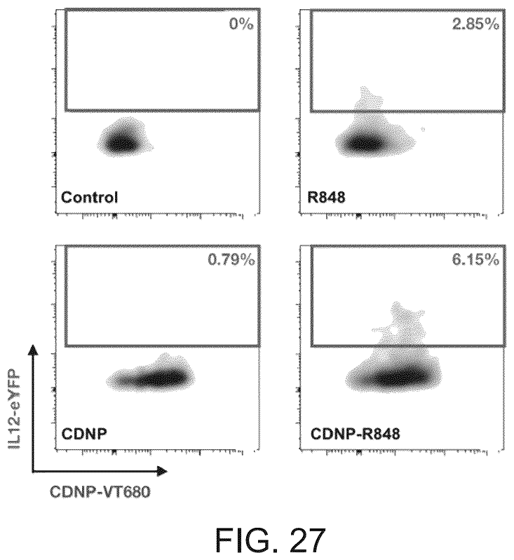

To examine macrophage re-education, both murine and human macrophages were polarized to an M2-state (IL-4 induction, 24 hrs) prior to drug treatment in the absence of IFNγ. For all experiments, dosing was matched (100 nM R848, 24 hr). FIG. 26A depicts qPCR assessment of transcription expressed as fold change relative to M2 (IL-4 treated) controls. For each gene on the x-axis, the left bar corresponds to R848 and the right bar corresponds to CDNP-R848. Data represent mean±s.d., N=3. CDNP-R848 treatment of murine macrophages enhances expression of M1-related genes (nos2, il 12, cd80) and further suppresses M2-related genes (mrc1, arg1), relative to treatment by R848 alone. FIG. 26B shows that M1-likeness is enhanced 5.9 fold following treatment by CDNP-R848 compared to R848 alone. Mean±s.e.m., N=3. P=0.08 (t-test). FIG. 26C shows expression of il12 by human macrophages treated by CDNP, R848, or CDNP-R848. M2-like and M1-like (LPS/INFγ treated) are provided for reference of baseline expression. Data represent mean±s.d. of fold change relative to β-actin, N=3. *P<0.05, **P<0.01, ***P<0.001 (ANOVA, Dunnett multiple comparison). These tests demonstrated enhanced M1-like transcription in murine and human M2 macrophages re-educated by R848, the transcription further enhanced by CDNP-R848.

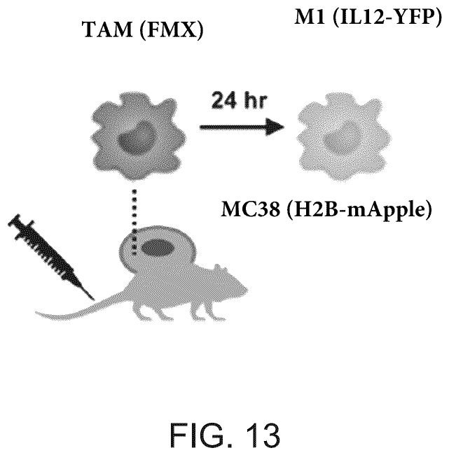

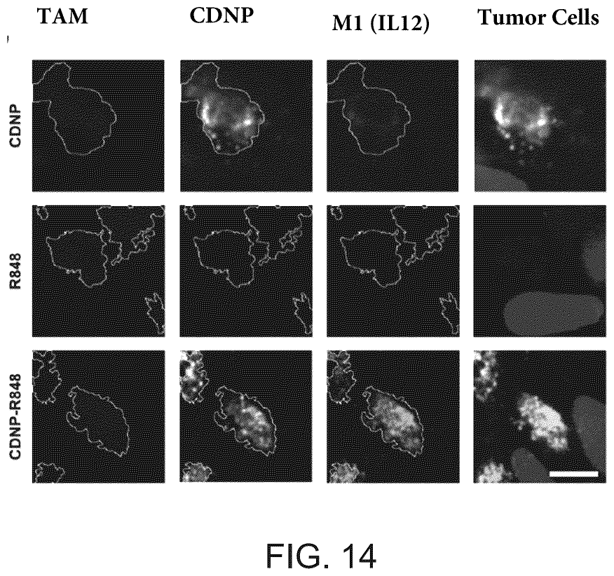

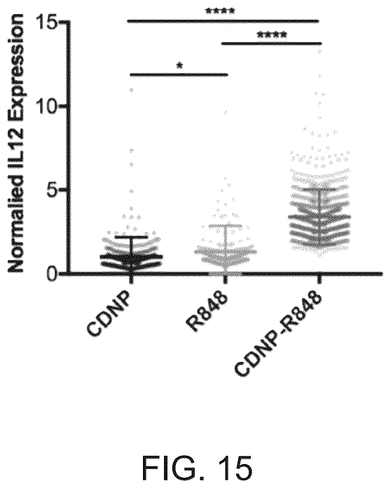

The pharmacodynamics of M1 induction in vivo were further explored by employing an IL12-YFP reporter mouse in which TAMs co-express YFP with the prototypical M1 marker IL12-p40. FIG. 13 is a diagram depicting intravital imaging of PacificBlue-ferumoxytol (FMX) labeled TAMs, CDNP-VT680, MC38-H2B-mApple tumor cells, and IL12-YFP expression by M1 macrophages. FIG. 14 depicts representative high magnification confocal fluorescence microscopy images of TAMs within tumors 24 hours following administration of CDNP (top row), R848 (middle row), or CDNP-R848 (bottom row). TAMs are outlined for clarity. Scale bar: 10 μm. FIG. 15 depicts quantified IL12 expression. Data represent mean±s.d., N>250 cells across 3 fields of view per condition. *P<0.05, ****P<0.0001 (ANOVA, Tukey HSD). According to this data, CDNP alone (without R848) accumulated in TAMs but did not elicit an IL12 response, and R848 itself also failed to elicit a robust IL12 response in vivo. In contrast, CDNP-R848, which showed potent accumulation in TAMs, induced a robust IL12 response.

FIG. 27 depicts representative flow cytometry plots of tumor associated macrophages (CD45+, F4/80+) obtained from IL12-eYFP mice bearing wild type intradermal MC38 tumors at 24 hours following i.v. administration of saline (control), R848, CDNP, or CDNP-R848. For CDNP-R848 treatments (bottom right), indicated regions of high CDNP-VT680 signal and high IL12 expression (gated, green) demonstrating CDNP-R848 uptake is associated with highly elevated IL12 production. This data independently confirmed IL12 induction by CDNP-R848, and this response was correlated with nanoparticle uptake by TAMs.

### Example 7. Therapeutic Efficacy

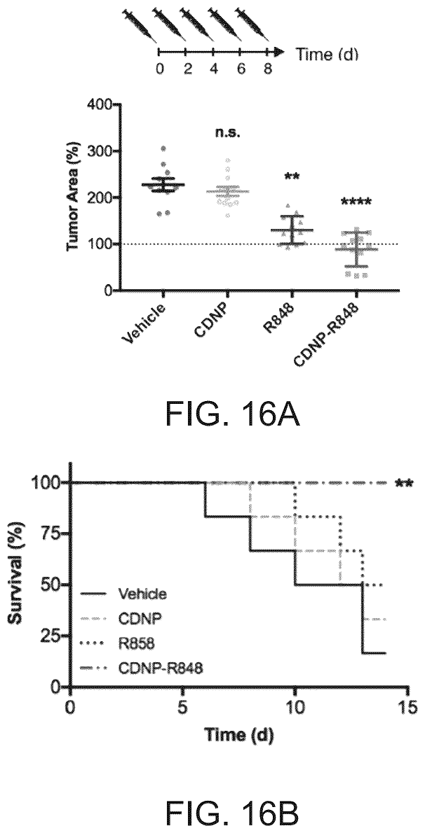

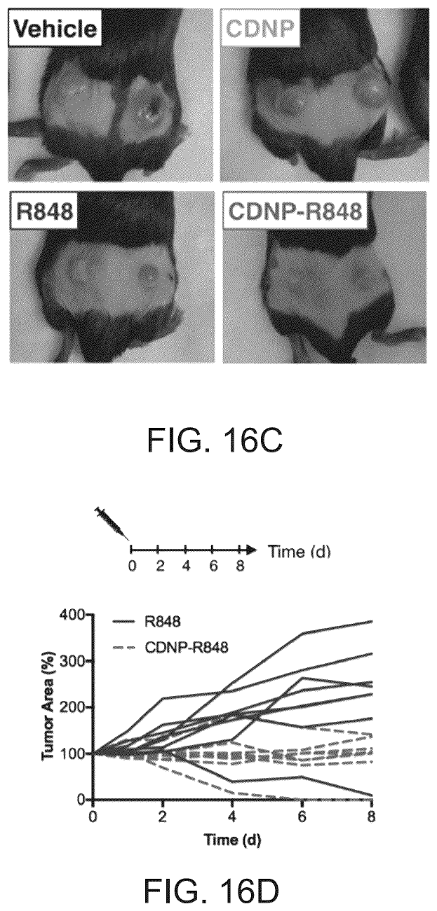

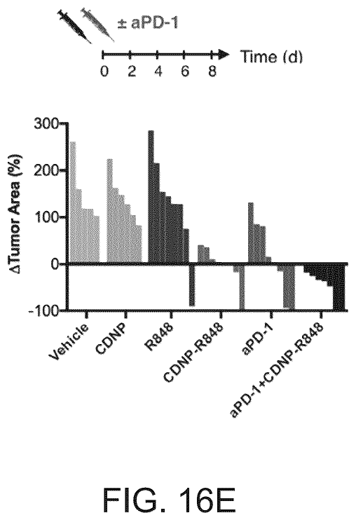

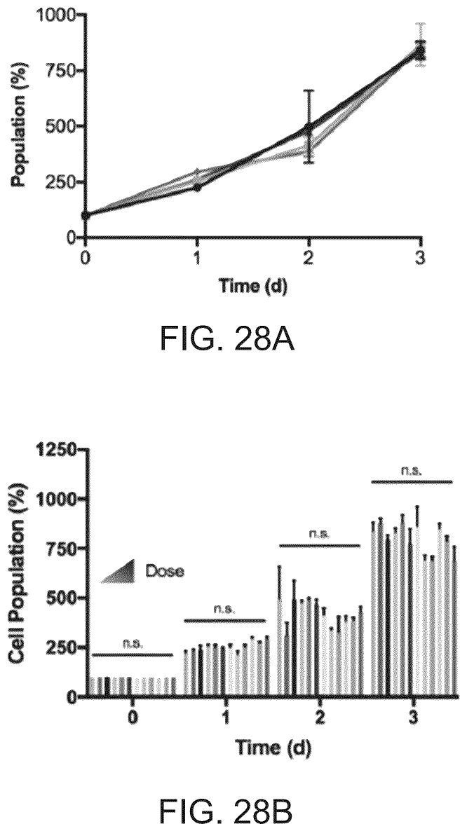

FIGS. 16A-16C are results of studies on the efficacy of repeated dosing regimen. FIG. 16A depicts tumor area at day 8 following the start of treatment. Data are expressed as mean±s.e.m; N=12; **P<0.01, ****P<0.0001 (Dunn's multiple comparison) relative to vehicle control. FIG. 16A shows that CDNP by itself did not have an effect on MC38 tumor growth relative to control animals. When given repeatedly in the free drug form, the small molecule R848 provided marginal benefits in terms of tumor control, not attributable to direct effects on tumor cell proliferation. To independently confirm this result, MC38 cell proliferation was monitored (PrestoBlue assay) in vitro in the presence of vehicle (DMSO) control, R848, CDNP or matched dose of CDNP-R848. FIG. 28A shows growth curves of a control (circles), R848 (triangles) (10 nM R848), CDNP (squares), and CDNP-R848 (diamonds), showing no effect on proliferation between groups. FIG. 28B shows that there was no change in cell population between groups at increased dose; first 3 bars in each group, control in increasing dose; second 3 bars in each group, R848 in increasing dose; third 3 bars in each group, CDNP in increasing dose; fourth 3 bars in each group, CDNP-R848 in increasing dose (10 nM, 100 nM, and 1.0 μM R848). P>0.1, two-way repeated measures ANOVA. Proliferation rate, determined by linear fit to each condition and dose, was unaltered by treatment conditions (P>0.96, F-test). Data represent mean±s.e.m. following normalization to baseline for each condition, N=4 per condition.

FIGS. 16B-16C further show that CDNP-R848 treated mice showed noticeably smaller tumors than in any other repeated treatment group, reduced tumor growth rates, and improved survival. FIG. 16B depicts a plot of survival vs. time following start of treatment. **P<0.01 (Log-rank test) relative to vehicle controls. FIG. 16C depicts macroscopic images of tumors at day 8 following initiation of treatment.

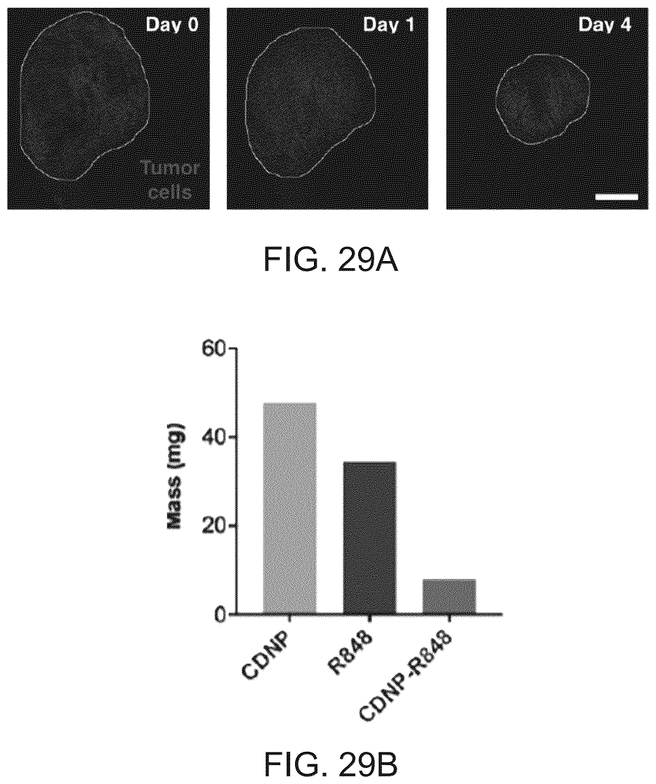

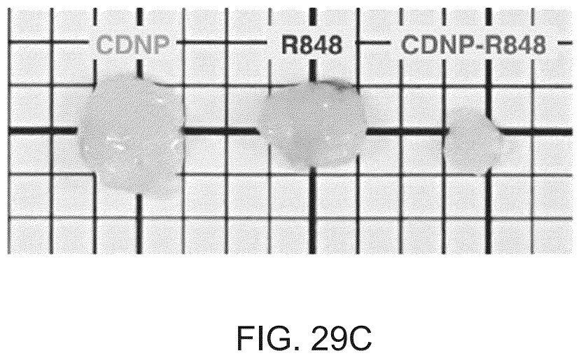

Observation of tumor recession for CDNP-R848 treatment was also observed. Tumor size was monitored following single-dose administration in IL12-YFP mice on day 0 (8 days following tumor inoculation). Treatment with single-dose CDNP-R848 (16.5 mg kg⁻¹ CDNP with 2.0 mg kg⁻¹ R848) resulted in rapid tumor recession, observed by confocal fluorescence microscopy of MC38-H2B-mApple tumors, outlined (FIG. 29A). Scale bar: 1.0 mm. Seven days after administration of R848 (2.0 mg kg⁻¹), CDNP (16.5 mg kg⁻¹), or CDNP (16.5 mg kg⁻¹) with R848 (2.0 mg kg⁻¹), relative tumor sizes were determined by tumor weight (FIG. 29B) and gross imaging (FIG. 29C) of resected tumor tissues. Drug nanoformulation resulted in smaller tumor size relative to CDNP and free drug controls, indicating feasibility of single-dose administration.

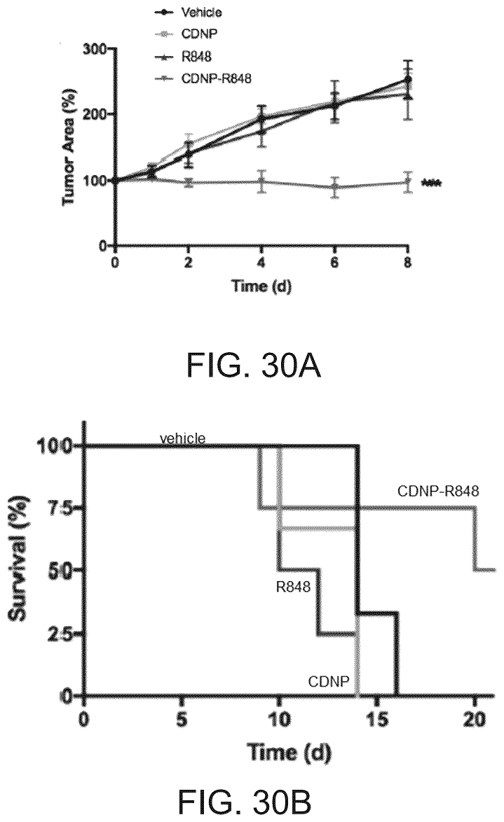

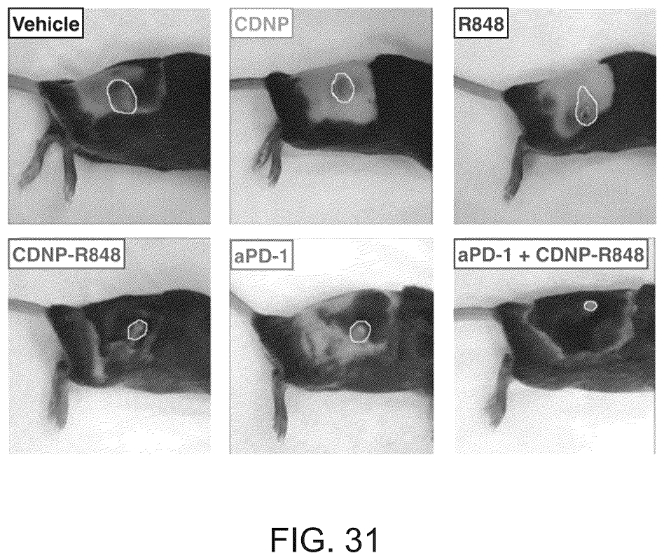

The tumor growth experiments were repeated using single dosage of free or nano-encapsulated R848, and it was found that CDNP assisted delivery of R848 to significantly improve therapeutic response (FIG. 16D), FIG. 16D depicts individual tumor growth curves for mice treated with a single dose of R848 or CDNP-R848. FIG. 30A depicts tumor growth curves following treatment. Data are expressed as mean±s.e.m; N≥6; **p<0.01 (Friedman, Dunn's multiple comparison) relative to vehicle control. FIG. 30B shows survival following start of treatment.

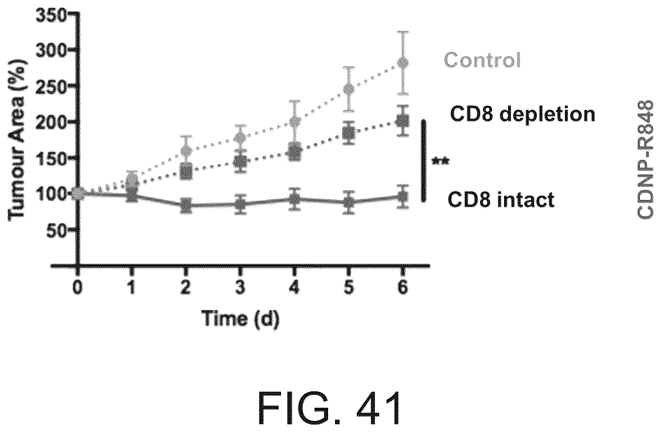

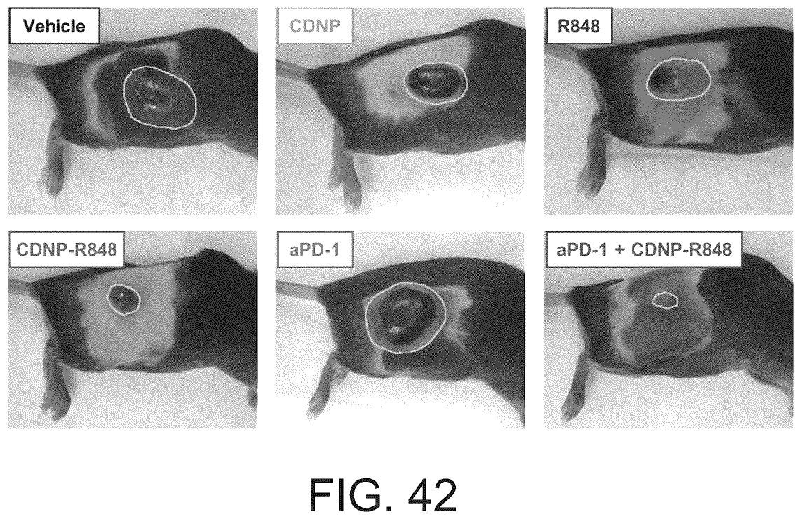

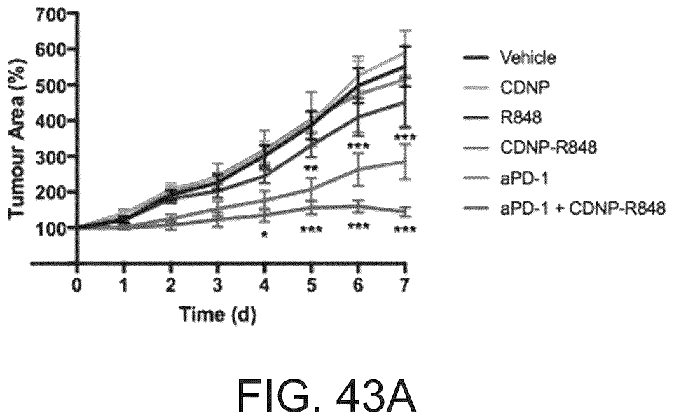

To examine the role of adaptive immune involvement, antibody depletion of CD8+T-cells was examined in treatment groups, relative to CDNP controls in C57BL/6 mice bearing MC38 tumours. FIG. 41 depicts tumor area vs. time for a vehicle control and CDNP-R848, with or without CD8 depletion or CD8 intact. Data are expressed as mean±s.e.m; N=8 tumours per treatment group, N=7 tumours for control; **P=0.004 (Friedman, Dunn's multiple comparison) relative to CDNP control. CDNP-R848 treatment in the absence of CD8±T-cells results in blunting but not complete elimination of treatment efficacy, indicating involvement of adaptive immunity.

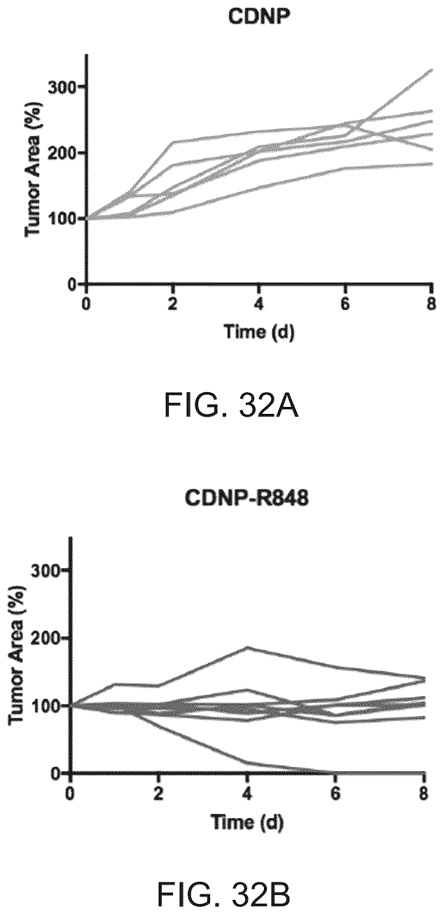

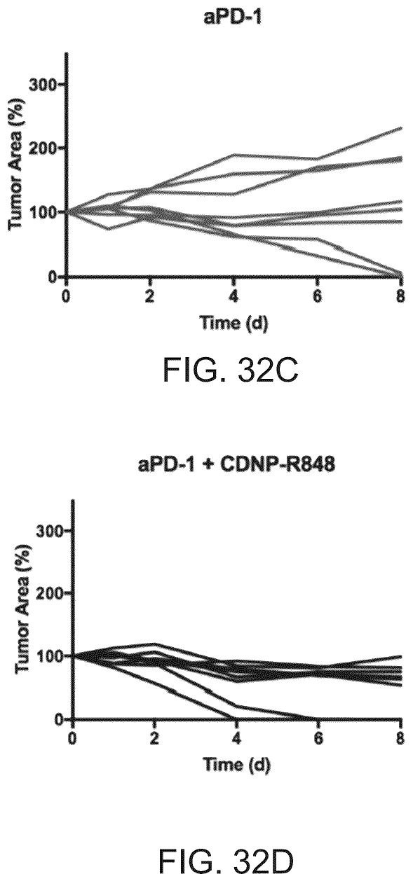

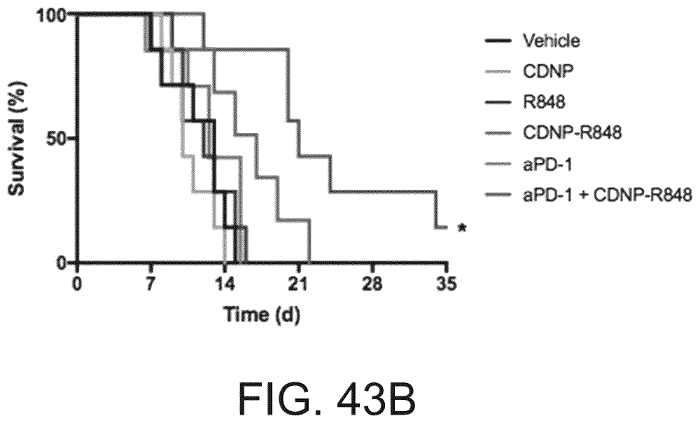

Given the productive diversion of TAMS from immune-suppressive to immune-supportive phenotypes and the demonstrated involvement of adaptive immunity, it is understood that CDNP-R848 monotherapy could potentiate checkpoint blockade. Combination of CDNP-R848 with anti-PD-1 was synergistic and resulted in tumor shrinkage, stabilization and homogenization of response, as shown in FIG. 16E. FIG. 16E depicts a bar graph of change in individual tumor area at day 8 following treatment with a single dose of CDNP, CDNP-R848, aPD-1 or the combination therapy. For all studies, treatment was initiated when tumors reached 25 mm². In addition, FIG. 31 shows images of mice that were acquired 7 days following administration of single agent (CDNP, CDNP-R848, aPD-1) or combination (aPD-1+CDNP-R848) therapy. Tumor margins are outlined. FIGS. 32A-32D depict individual MC38 tumor growth curves in response to single agent (CDNP, CDNP-R848, aPD-1) or combination (aPD-1+CDNP-R848) therapy. Heterogeneity of aPD-1 response is reduced by combination with CDNP-R848, and results in rapid tumor regression in some cases. Complete tumor regression was observed in 2/7 animals. These results support the synergistic combination of immunotherapies that target both innate and adaptive immune response to improve therapeutic efficacy. Similar results were observed in B16F10 melanoma, which is otherwise unresponsive to aPD-1 treatment alone. FIG. 43 depicts the tumor area vs. time and survival of mice bearing B16F10 tumors after treatment with a vehicle, CDNP, R848, CDNP-R848, aPD-1, and aPD-1+CDNP-R848.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims,

What is claimed is:

1. A nanoparticle, comprising at least two host macrocycles, wherein the at least two host macrocycles are covalently crosslinked by a linker, wherein the linker comprises a moiety of Formula (I): 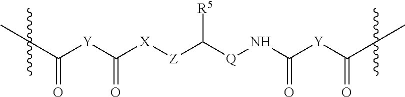
wherein:
Q is selected from a bond or methylene;
X is selected from O, S, and NR¹;
each Y is independently selected from C₁₋₁₀ alkylene optionally substituted with one or more R²;
Z is selected from A-B, wherein A is selected from a bond and C₁₋₁₀ alkylene, and B is selected from C₁₋₁₀ arylene, 3-10 membered heteroarylene, 3-10 membered heterocycloalkyl, and C₃₋₁₀ cycloalkyl;
wherein A is optionally substituted with one or more R³, and B is optionally substituted with one or more R⁴;
R¹ is selected from H and C₁₋₃ alkyl;
each R² is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
each R⁴ is independently selected from C₁₋₁₀ arylene, 3-10 membered heteroarylene, 3-10 membered heterocycloalkyl, C₃₋₁₀ cycloalkyl, hydroxy, halo, CN, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and
R⁵ is selected from H, C₁-C₆ alkyl, CO₂H, C₁₋₁₀ arylene, 3-10 membered heteroarylene, 3-10 membered heterocycloalkyl, C₃₋₁₀ cycloalkyl, hydroxy, halo, CN, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl.

2. The nanoparticle of claim 1, wherein R⁵ is CO₂H.

3. The nanoparticle of any one of the preceding claims, wherein Q is a bond.

4. The nanoparticle of any one of the preceding claims, wherein each Y is ethylene.

5. The nanoparticle of any one of the preceding claims, wherein X is NH.

6. The nanoparticle of any one of the preceding claims, wherein Z is n-butylene.

7. The nanoparticle of any one of the preceding claims, wherein the at least two host macrocycles comprise less than 1×10⁹ host macrocycles.

8. The nanoparticle of any one of the preceding claims, wherein the at least two host macrocycles comprise less than 5×10⁶ host macrocycles.

9. The nanoparticle of any one of the preceding claims, wherein the at least two host macrocycles comprise less than 5000 host macrocycles.

10. The nanoparticle of any one of the preceding claims, wherein at least one of the at least two host macrocycles is selected from the group consisting of: cyclodextrin, pillar[n]arenes, calix[n]arenes, and cucurbit[n]urils.

11. The nanoparticle of any one of the preceding claims, wherein at least two of the at least two host macrocycles are selected from the group consisting of: cyclodextrin, pillar[n]arenes, calix[n]arenes, and cucurbit[n]utils.

12. The nanoparticle of any one of claims 1-9, wherein the at least two host macrocycles comprise at least two cyclodextrins.

13. The nanoparticle of claim 12, wherein each cyclodextrin comprises α-cyclodextrin, β-cyclodextrin, γ-cyclodextrin, 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-65 -cyclodextrin, a cyclodextrin sulfobutylether, a cyclodextrin thioether, a cyanoethylated cyclodextrin, a succinyl-cyclodextrin, or an aminated cyclodextrin.

14. The nanoparticle of any one of claims 12-13, wherein each cyclodextrin comprises β-cyclodextrin.

15. The nanoparticle of any one of the preceding claims, wherein the nanoparticle comprises at least one linear or branched polymer.

16. The nanoparticle of claim 15, wherein the at least one polymer is selected from the group consisting of: a dextran derivative, a hyaluronic acid derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative, a oligo(poly(ethylene glycol)fumarate) derivative, a poly(vinyl alcohol) derivative, and a poly(vinyl acid) derivative.

17. The nanoparticle of any one of the preceding claims, wherein the nanoparticle comprises at least one therapeutic agent.

18. The nanoparticle of claim 17, wherein the therapeutic agent forms a host-guest complex with at least one of the host macrocycles.

19. The nanoparticle of any one of claims 17-18, wherein the at least one therapeutic agent comprises an anticancer or immunomodulating agent.

20. The nanoparticle of any one of claims 17-19, wherein the at least one therapeutic agent comprises an anticancer agent.

21. The nanoparticle of claim 20, wherein the anticancer agent is a toll-like receptor (TLR) agonist.

22. The nanoparticle of claim 20, wherein the anticancer agent is a TLR3, TLR4, TLR 7/8, or TLR9 agonist.

23. The nanoparticle of any one of claims 17-18, wherein one or more of the at least one therapeutic agents is selected from the group consisting of: GW2580, CEP32496, BLZ945, OSI930, PLX3397, dasatinib, sunitinib, ABT869, imatinib, foretinib, XL228, gefitinib, PD0325901, trametinib, bentamapimod, dabrafenib, vemurafinib, crizotinib, UNC2025, indoximod, celecoxib, rapamycin, N1K12192, trichostatin A, IBET151, TMP195, BYL719, GDC0941, BKM120, imiquimod, gardiquimod, resiquimod (R848), motolimod, and GS9620.

24. The nanoparticle of any one of claims 17-18 and 23, wherein one or more of the at least one therapeutic agents is a compound selected from the group consisting of: imiquimod, indoximod, gardiquimod, motolimod, and resiquimod (R848).

25. The nanoparticle of any one of claims 20-22, wherein the anticancer agent is resiquimod (R848),

26. The nanoparticle of any one of claims 17-25, wherein the nanoparticle comprises two or more therapeutic agents, wherein one of the two or more therapeutic agents improves the efficacy of one or more of the other therapeutic agents.

27. The nanoparticle of any one of the preceding claims, wherein the nanoparticle further comprises an imaging agent.

28. The nanoparticle of claim 27, wherein the imaging agent comprises a magnetic resonance imaging (MRI) agent, a positron emission tomography (PET) agent, a single-photon emission computed tomography (SPECT) agent, or a near-infrared fluorophore.

29. The nanoparticle of claim 28, wherein the near-infrared fluorophore is selected from the group consisting of Vivo Tag 680-XL, ZW800-IC, ZW800-1, ZW800-3C, ZW700-1, indocyanine green (ICG), Cy5, Cy5.5, Cy7, Cy7.5, IRDye800-CW (CW800), BODIPY 630, and ZWCC.

30. The nanoparticle of any one of claims 17-29, wherein the at least one therapeutic agent is conjugated with a fluorescent dye.

31. The nanoparticle of any one of claims 17-30, wherein the at least one therapeutic agent is conjugated with adamantane.

32. The nanoparticle of any one of claims 17-31, wherein the stoichiometric ratio of the cyclodextrin to the therapeutic agent is from about 100:1 to about 1:100.

33. The nanoparticle of any one of claims 17-31, wherein the stoichiometric ratio of the cyclodextrin to the therapeutic agent is about 1:1.

34. The nanoparticie of any one of claims 17-31, wherein the stoichiometric ratio of the cyclodextrin to the therapeutic agent is about 1.1:1.

35. The nanoparticle of any one of claims 17-34, wherein the equilibrium binding constant (K_{D}) of the at least one therapeutic agent to the host macrocycle is from about 1×10⁻¹² M to about 0.1 M.

36. The nanoparticle of any one of claims 17-34, wherein the equilibrium binding constant (K_{D}) of the at least one therapeutic agent to the cyclodextrin is from about 5.5 mM to about 7.2 mM.

37. The nanoparticle of any one of claims 17-34, wherein the equilibrium binding constant (K_{D}) of the at least one therapeutic agent to the cyclodextrin is about 6.3 mM.

38. The nanoparticle of any one of claims 17-37, wherein the half-life of the therapeutic agent in vivo after release from the nanoparticle is from about 45 minutes to about 90 minutes.

39. The nanoparticle of any one of claims 17-37, wherein the half-life of the therapeutic agent in vivo after release from the nanoparticle is about 62 minutes.

40. The nanoparticle of any one of claims 1-39, wherein the nanoparticle has an overall negative charge.

41. The nanoparticle of any one of claims 1-40, wherein the nanoparticle has a zeta potential of from about −5 mV to about −50 mV.

42. The nanoparticle of any one of claims 1-40, wherein the nanoparticle has a zeta potential of about −10 mV.

43. The nanoparticle of any one of claims 1-42, wherein the average molecular weight of the nanoparticle is from about 1,500 g/mol to about 5×10¹¹ g/mol.

44. The nanoparticle of any one of claims 1-42, wherein the average molecular weight of the nanoparticle is from about 15×10³ g/mol to about 20×10⁶ g/mol.

45. The nanoparticle of any one of claims 1-42, wherein the average molecular weight of the nanoparticle is about 20×10⁶ g/mol.

46. The nanoparticle of any one of claims 1-45, wherein the nanoparticle comprises an average of from about 10 to about 10,000 cyclodextrins.

47. The nanoparticle of any one of claims 1-45, wherein the nanoparticle comprises an average of from about 100 to about 2,000 cyclodextrins.

48. The nanoparticle of any one of claims 1-45, wherein the nanoparticle comprises an average of about 1,000 cyclodextrins.

49. The nanoparticle of any one of claims 1-48, wherein the average hydrodynamic diameter of the nanoparticle is from about 10 nm to about 1000 nm.

50. The nanoparticle of any one of claims 1-48, wherein the average hydrodynamic diameter of the nanoparticle is from about 10 nm to about 70 nm.

51. The nanoparticle of any one of claims 1-48, wherein the average hydrodynamic diameter of the nanoparticle is from about 20 nm to about 60 nm.

52. The nanoparticle of any one of claims 1-48, wherein the average hydrodynamic diameter of the nanoparticle is about 50 nm.

53. The nanoparticle of any one of claims 1-48, wherein the average hydrodynamic diameter of the nanoparticle is about 30 nm.

54. A nanoparticle, comprising:
at least two cyclodextrins, wherein the at least two cyclodextrins are covalently crosslinked by a linker, and wherein the linker comprises a moiety of Formula (I) 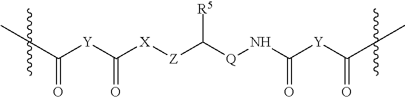
wherein:
Q is selected from a bond or methylene;
X is selected from 0, 8, and NR¹;
each Y is independently selected from C₁₋₁₀ alkylene optionally substituted with one or more R²;
Z is selected from A-B, wherein A is selected from a bond and C₁₋₁₀ alkylene, and B is selected from C₁₋₁₀ arylene, 3-10 membered heteroarylene, 3-10 membered heterocycloalkyl, and C₃₋₁₀ cycloalkyl;
wherein A is optionally substituted with one or more R₃, and B is optionally substituted with one or more R⁴;
R¹ is selected from H and C₁₋₃ alkyl;
each R² is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
each R³ is independently selected from C₁₋₁₀ arylene, 3-10 membered heteroarylene, 3-10 membered heterocycloalkyl, C₃₋₁₀ cycloalkyl, hydroxy, halo, CN, oxo, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl,
each R⁴ is independently selected from C₁₋₁₀ arylene, 3-10 membered heteroarylene, 3-10 membered heterocycloalkyl, C₃₋₁₀ cycloalkyl, hydroxy, halo, CN, C₁-C₆ alkyl, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and
R⁵ is selected from H, C₁-C₆ alkyl, CO₂H, C₁₋₁₀ arylene, 3-10 membered heteroarylene, 3-10 membered heterocycloalkyl, C₃₋₁₀ cycloalkyl, hydroxy, halo, CN, C₁-C₆ alkoxy, NH₂, COOC₁-C₆ alkyl, CONH₂, CONHC₁-C₆ alkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl; and
a therapeutic agent.

55. The nanoparticle of claim 54, wherein R⁵ is CO₂H.

56. The nanoparticle of any one of claims 54-55, wherein Q is a bond.

57. The nanoparticle of any one of claims 54-56, wherein each Y is ethylene.

58. The nanoparticle of any one of claims 54-57, wherein X is NH.

59. The nanoparticle of any one of claims 54-58, wherein Z is n-butylene.

60. The nanoparticle of any one of claims 54-59, wherein the at least two host macrocycles comprise less than ×10⁹ host macrocycles.

61. The nanoparticle of any one of claims 54-59, wherein the at least two host macrocycles comprise less than 5×10⁶ host macrocycles.

62. The nanoparticle of any one of claims 54-59, wherein the at least two host macrocycles comprise less than 5000 host macrocycles.

63. The nanoparticle of any one of claims 54-62, wherein each cyclodextrin comprises α-cyclodextrin, β-cyclodextrin, γ-cyclodextrin, 2-hydroxypropyl-α-cyclodextrin, 2-hydroxypropyl-β-cyclodextrin, 2-hydroxypropyl-γ-cyclodextrin, methyl-α-cyclodextrin, methyl-β-cyclodextrin, methyl-γ-cyclodextrin, a cyclodextrin sulfobutylether, a cyclodextrin thioether, a cyanoethylated cyclodextrin, a succinyl-cyclodextrin, or an aminated cyclodextrin.

64. The nanoparticle of any one of claims 54-62, wherein each cyclodextrin comprises β-cyclodextrin.

65. The nanoparticle of any one of claims 54-64, wherein the linker comprises L-lysine.

66. The nanoparticle of any one of claims 54-65, wherein the nanoparticle comprises at least one linear or branched polymer.

67. The nanoparticle of claim 66, wherein the at least one polymer is selected from the group consisting of: a dextran derivative, a hyaluronic acid derivative, a chitosan derivative, a fucoidan derivative, an alginate derivative, a cellulose derivative, a collagen derivative, a poly(ethylene glycol) derivative, a poly(hydroxyethyl acrylate) derivative, a poly(hydroxyethyl methacrylate) derivative, a poly(N-isopropylacrylamide) derivative, a poly(glycolic acid), a poly(lactic acid) derivative, a poly(lactic acid-glycolic acid) derivative, a oligo(poly(ethylene glycol)fumarate) derivative, a poly(vinyl alcohol) derivative, and a poly(vinyl acid) derivative.

68. The nanoparticle of any one of claims 54-67, wherein the therapeutic agent forms a host-guest complex with at least one of the cyclodextrins.

69. The nanoparticle of any one of claims 54-68, wherein the at least one therapeutic agent comprises an anticancer agent.

70. The nanoparticle of claim 69, wherein the anticancer agent is a toll-like receptor (TLR) agonist.

71. The nanoparticle of claim 70, wherein the anticancer agent is a TLR 7/8 agonist.

72. The nanoparticle of any one of claims 54-68, wherein one or more of the at least one therapeutic agents is selected from the group consisting of: GW2580, CEP32496, BLZ945, OSI930, PLX3397, dasatinib, sunitinib, ABT869, imatinib, foretinib, XL228, gefitinib, PD0325901, trametinib, bentamapimod, dabrafenib, vemurafinib, crizotinib, UNC2025, indoximod, celecoxib, rapamycin, NIK12192, trichostatin A, IBET151, TMP195, BYL719, GDC0941, BKM120, resiquimod (R848), motolimod, GS9620, and a compound comprising an imidazoquinoline.

73. The nanoparticle of any one of claims 54-68, wherein one or more of the at least one therapeutic agents is a compound selected from the group consisting of: imiquimod, indoximod, gardiquimod, motolimod, or resiquimod (R848).

74. The nanoparticle of any one of claims 54-68, wherein one or more of the at least one therapeutic agents is resiquimod (R848).

75. The nanoparticle of any one of claims 54-74, wherein the nanoparticle comprises two or more therapeutic agents, wherein one of the two or more therapeutic agents improves the efficacy of one or more of the other therapeutic agents.

76. The nanoparticle of any one of claims 54-75, wherein the nanoparticle further comprises an imaging agent.

77. The nanoparticle of claim 76, wherein the imaging agent comprises a magnetic resonance imaging (MRI) agent, a positron emission tomography (PET) agent, a single-photon emission computed tomography (SPECT) agent, or a near-infrared fluorophore.

78. The nanoparticle of claim 77, wherein the near-infrared fluorophore is selected from the group consisting of Vivi Tag 680-XL, ZW800-1C, ZW800-1, ZW800-3C, ZW700-1, indocyanine green (ICG), Cy5, Cy5.5, Cy7, Cy7.5, IRDye800-CW (CW800), BODIPY 630, and ZWCC.

79. The nanoparticle of any one of claims 54-78, wherein the at least one therapeutic agent is conjugated with a fluorescent dye.

80. The nanoparticle of any one of claims 54-79, wherein the at least one therapeutic agent is conjugated with adamantine.

81. The nanoparticle of any one of claims 54-80, wherein the stoichiometric ratio of the cyclodextrin to the therapeutic agent is from about 100:1 to about 1:100.

82. The nanoparticle of any one of claims 54-80, wherein the stoichiometric ratio of the cyclodextrin to the therapeutic agent is about 1:1.

83. The nanoparticle of any one of claims 54-80, wherein the stoichiometric ratio of the cyclodextrin to the therapeutic agent is about 1.1:1.

84. The nanoparticle of any one of claims 54-83, wherein the equilibrium binding constant (K_{D}) of the at least one therapeutic agent to the cyclodextrin is from about 1×10⁻¹² M to about 0.1 M.

85. The nanoparticle of any one of claims 54-83, wherein the equilibrium binding constant (K_{D}) of the at least one therapeutic agent to the cyclodextrin is from about 5.5 mM to about 7.2 mM.

86. The nanoparticle of any one of claims 54-83, wherein the equilibrium binding constant (K_{D}) of the at least one therapeutic agent to the cyclodextrin is about 6.3 mM.

87. The nanoparticle of any one of claims 54-86, wherein the half-life of the therapeutic agent in vivo after release from the nanoparticle is from about 45 minutes to about 90 minutes.

88. The nanoparticle of any one of claims 54-86, wherein the half-life of the therapeutic agent in vivo after release from the nanoparticle is about 62 minutes.

89. The nanoparticle of any one of claims 54-88, wherein the nanoparticle has an overall negative charge.

90. The nanoparticle of any one of claims 54-89, wherein the nanoparticle has a zeta potential of from about −5 mV to about −15 mV.

91. The nanoparticle of any one of claims 54-89, wherein the nanoparticle has a zeta potential of about −10 mV.

92. The nanoparticle of any one of claims 54-91, wherein the average molecular weight of the nanoparticle is from about 1,500 g/mol to about 5×10¹¹ g/mol.

93. The nanoparticle of any one of claims 54-91, wherein the average molecular weight of the nanoparticle is from about 15×10³ g/mol to about 20×10⁶ g/mol.

94. The nanoparticle of any one of claims 54-91, wherein the average molecular weight of the nanoparticle is about 20×10⁶ g/mol.

95. The nanoparticle of any one of claims 54-94, wherein the nanoparticle comprises an average of from about 10 to about 10,000 cyclodextrins.

96. The nanoparticle of any one of claims 54-94, wherein the nanoparticle comprises an average of from about 100 to about 2,000 cyclodextrins.

97. The nanoparticle of any one of claims 54-94, wherein the nanoparticle comprises an average of about 1,000 cyclodextrins.

98. The nanoparticle of any one of claims 54-97, wherein the average hydrodynamic diameter of the nanoparticle is from about 10 nm to about 1000 nm.

99. The nanoparticle of any one of claims 54-97, wherein the average hydrodynamic diameter of the nanoparticle is from about 10 nm to about 70 nm.

100. The nanoparticle of any one of claims 54-97, wherein the average hydrodynamic diameter of the nanoparticle is from about 20 nm to about 60 nm.

101. The nanoparticle of any one of claims 54-97, wherein the average hydrodynamic diameter of the nanoparticle is about 50 nm.

102. The nanoparticle of any one of claims 54-97, wherein the average hydrodynamic diameter of the nanoparticle is about 30 nm.

103. A pharmaceutical composition comprising the nanoparticle of any one of claims 17-102 and a pharmaceutically acceptable excipient.

104. A method of treating cancer in a patient, the method comprising administering a therapeutically effective amount of the nanoparticle of any one of claims 17-102, or the pharmaceutical composition of claim 103, to the patient.

105. The method of claim 104, wherein the cancer comprises a tumor-associated macrophage, and wherein the phenotype of the macrophage is M2.

106. The method of claim 105, wherein the treating further comprises converting the phenotype of the macrophage from M2 to M1.

107. The method of any one of claims 104-106, wherein the cancer is selected from the group consisting of Ewing sarcoma, osteosarcoma, glioblastoma, meningioma, oligodendrial cancer, melanoma metastasis, melanoma primary, breast cancer, gastric cancer, germ cell tumors, astrocytoma, ovarian cancer, lung large cell carcinoma, lung adenocarcinoma, lung squamous cell carcinoma, colon cancer, head and neck cancer, bladder cancer, thyroid cancer, liver cancer, pancreas cancer, kidney cancer, cervical cancer, testicular cancer, prostate cancer, and bone cancer.

108. The method of any one of claims 104-107, wherein the cancer is metastatic.

109. The method of any one of claims 104-108, wherein the uptake of the nanoparticle is higher into tumor associated macrophages than into any other organ or tissue type in the subject after administration.

110. The method of any one of claims 104-109, wherein less than 20 mol % of the therapeutic agent is released prior to uptake of the nanoparticle into tumor macrophage cells.

111. The method of any one of claims 104-109, wherein less than 10 mol % of the nanoparticle is released prior to uptake of the nanoparticle into tumor macrophage cells.

112. The method of any one of claims 104-109, wherein less than 5 mol % of the nanoparticle is released prior to uptake of the nanoparticle into tumor macrophage cells.

113. The method of any one of claims 104-109, wherein less than 1 mol % of the nanoparticle is released prior to uptake of the nanoparticle into cancer cells.

114. The method of any one of claims 104-113, wherein the nanoparticle or composition is administered intravenously, intraarterially, intratumorally, subcutaneously, or intraperitoneally.

115. The method of any one of claims 104-114, further comprising administering an additional therapeutic agent that improves the efficacy of the nanoparticle.

116. The method of claim 115, wherein the additional therapeutic agent is a PD-1 antibody, a CTLA-4 antibody, a PD-L1 antibody, an IDO inhibitor, a CSF-1R inhibitor, kinase inhibitor, an MAC inhibitor, a PI3K inhibitor, a MerTK inhibitor, or an Ax1 inhibitor.

117. The method of any one of claims 115-116, wherein the additional therapeutic agent is a antibody.

118. The method of claim 117, wherein the PD-1 antibody is selected from the group consisting of: nivolumab, pembrolizumab, pidilizumab, BMS-936559, atezolizumab, and avelumab.

119. The method of any one of claims 104-118, further comprising treating the patient with radiation, chemotherapy, antibody checkpoint therapy, immunotherapy, or any, combination thereof.

120. The method of any one of claims 104-119, wherein the treating comprises slowing the formation of cancer cells.

121. The method of any one of claims 104-120, wherein the treating comprises preventing the formation of cancer cells.

122. The method of any one of claims 104-121, wherein the treating comprises killing cancer cells.

123. The method of any one of claims 104-122, wherein the patient is a human.

124. A method of altering the phenotype of a tumor-associated macrophage in a cancer cell, comprising contacting the anticancer agent of the nanoparticle of any one of claims 20-102 with the cancer cell.

125. The method of claim 124, wherein the altering comprises converting an M2 phenotype to an M1 phenotype.

126. A method of reducing the toxicity, side effects, or both of a chemotherapeutic agent in a patient, comprising administering a therapeutically effective amount of the nanoparticle of any one of claims 17-102, or the pharmaceutical composition of claim 103 to the patient.

127. The method of claim 126, wherein the chemotherapeutic agent is administered systemically, and comprises a TLR7/8 inhibitor.

128. The method of claim 126, wherein the TLR7/8 inhibitor comprises resiquimod (R848).
